(19) United States
(12) Patent Application Publication
(10) Pub. No.: US 2026/0263813 A1
(43) Pub. Date: Sep. 10, 2026

(54) PROSTHETIC VALVE PACING AND SENSING SYSTEMS

(71) Applicant: SMARTVALVE SYSTEMS LTD., Savyon (IL)

(72) Inventors: Yossi GROSS, Moshav Mazor (IL); Navot Rabban, Ramat Gan (IL); Meni Iamberger, Kfar Saba (IL)

(73) Assignee: SMARTVALVE SYSTEMS LTD., Savyon (IL)

(21) Appl. No.: 19/662,286

(22) Filed: Apr. 29, 2026

Related U.S. Application Data
(63) Continuation of application No. 19/381,343, filed on Nov. 6, 2025.
(63) Continuation-in-part of application No. 19/466,635, filed on Feb. 2, 2026.
Application No. 19/466,635 is a continuation of application No. PCT/IL2024/050830, filed on Aug. 18, 2024.
Application No. PCT/IL2024/050830 is a continuation-in-part of application No. 18/607,638, filed on Mar. 18, 2024.
Application No. 18/607,638 is a continuation-in-part of application No. 18/452,229, filed on Aug. 18, 2023, now Pat. No. 11,931,255.
Application No. 18/452,229 is a continuation-in-part of application No. 18/452,216, filed on Aug. 18, 2023, now Pat. No. 11,975,203.
(60) Provisional application No. 63/717,923, filed on Nov. 8, 2024.
(60) Provisional application No. 63/809,535, filed on May 21, 2025.
(60) Provisional application No. 63/849,212, filed on Jul. 23, 2025.
(60) Provisional application No. 63/849,229, filed on Jul. 23, 2025.

(51) Int. Cl. A61N 1/362; A61F 2/24; A61N 1/05

(52) U.S. Cl. CPC A61N 1/362; A61F 2/2418; A61N 1/056

(57) ABSTRACT

A prosthetic cardiac valve system is provided that includes a prosthetic cardiac valve, which includes a frame, which includes interconnected stent cells, which include distal stent cells that are located in a distal half of the frame and are shaped so as to define respective distal peaks; prosthetic leaflets coupled to the frame; and electrodes, which include a plurality of distal electrodes mechanically coupled to the frame at or near respective ones of the distal peaks. Circuitry is electrically coupled to the electrodes, and is configured to apply pacing to the heart using one or more of the distal electrodes as one or more anodes and one or more of the other distal electrodes as one or more cathodes. Other embodiments are also described.

### CROSS-REFERENCE TO RELATED APPLICATIONS

The present application is:
(a) a continuation-in-part of U.S. application Ser. No. 19/381,343, filed Nov. 6, 2025, which claims the benefit of (i) U.S. Prov. Appl. 63/717,923, filed Nov. 8, 2024, (ii) U.S. Prov. Appl. 63/849,212, filed Jul. 23, 2025, (iii) U.S. Prov. Appl. 63/809,535, filed May 21, 2025, and (iv) U.S. Prov. Appl. 63/849,229, filed Jul. 23, 2025,
(b) a continuation-in-part of U.S. application Ser. No. 19/466,635, filed Feb. 2, 2026, which is a continuation of International Appl. PCT/IL2024/050830, filed Aug. 18, 2024, which published as PCT Publication WO 2025/041129 to Gross et al. and which is a continuation-in-part of U.S. application Ser. No. 18/607,638, filed Mar. 18, 2024, which published as U.S. Patent Application Publication US 2025/0058124 to Gross et al. and is (i) a continuation-in-part of U.S. application Ser. No. 18/452,229, filed Aug. 18, 2023, now U.S. Pat. No. 11,931,255, and (ii) a continuation-in-part of U.S. application Ser. No. 18/452,216, filed Aug. 18, 2023, now U.S. Pat. No. 11,975,203, and
(c) a continuation-in-part of U.S. application Ser. No. 19/409,867, filed Dec. 5, 2025, which is a continuation-in-part of International Appl. PCT/IL2024/050830, filed Aug. 18, 2024, which published as PCT Publication WO 2025/041129 to Gross et al. and which is a continuation-in-part of U.S. application Ser. No. 18/607,638, filed Mar. 18, 2024, which published as U.S. Patent Application Publication US 2025/0058124 to Gross et al. and is (i) a continuation-in-part of U.S. application Ser. No. 18/452,229, filed Aug. 18, 2023, now U.S. Pat. No. 11,931,255, and (ii) a continuation-in-part of U.S. application Ser. No. 18/452,216, filed Aug. 18, 2023, now U.S. Pat. No. 11,975,203.

All of the above-referenced applications are assigned to the assignee of the present application and incorporated herein by reference.

### FIELD OF THE APPLICATION

The present invention relates generally to surgical implants and systems, and specifically to prosthetic aortic valves and systems.

### BACKGROUND OF THE APPLICATION

Aortic heart valve replacement may be necessary to treat valve regurgitation or stenotic calcification of the leaflets. In percutaneous transluminal delivery techniques, a prosthetic aortic valve is compressed for delivery in a catheter and advanced through the descending aorta to the heart, where the prosthetic valve is deployed in the aortic valve annulus. New-onset cardiac conduction disturbances are common after transcatheter aortic valve replacement (TAVR). The most common complication is left bundle branch block (LBBB).

PCT Publication WO 2022/149130 to Gross, which is incorporated herein by reference, inter alia describes a prosthetic aortic valve, which is configured to be delivered to a native aortic valve of a patient in a constrained delivery configuration within a delivery sheath. The prosthetic aortic valve includes a frame, which includes interconnected stent struts arranged so as to define interconnected stent cells; a plurality of prosthetic leaflets coupled to the frame; a cathode and an anode, which are mechanically coupled to the frame; and a prosthetic-valve coil, which is in non-wireless electrical communication with the cathode and the anode, and is coupled to a plurality of the stent struts, running along the stent struts so as to surround a plurality of the stent cells when the prosthetic aortic valve is in an expanded fully-deployed configuration upon release from the delivery sheath.

US Patent Application Publication 2017/0258585 to Marquez et al. describes sensor-integrated prosthetic valves that can comprise a variety of features, including a plurality of valve leaflets, a frame assembly configured to support the plurality of valve leaflets and define a plurality of commissure supports terminating at an outflow end of the prosthetic valve, a sensor device associated with the frame assembly and configured to generate a sensor signal, for example, a sensor signal indicating deflection of one or more of the plurality of commissure supports, and a transmitter assembly configured to receive the sensor signal from the sensor device and wirelessly transmit a transmission signal that is based at least in part on the sensor signal.

U.S. Pat. No. 9,326,854 to Casley et al. describes medical device delivery assemblies. The assembly may include a catheter-based delivery system. The assembly may include a pacing element to pace a patient's heart before, during, or after a procedure. The pacing element may be a detachable, implanting pacing element. The pacing element may be an implantable pacemaker and the implantable pacemaker may be disposed on a catheter-based delivery system. The assembly may include a prosthetic heart valve with one or more pacing elements on it. The pacing element may include a pacing strip or strips. These strips may be conductive or insulative. These strips may prevent, treat, or correct abnormal electrical communication in a heart.

U.S. Pat. No. 11,331,476 to Capek et al. describes a method including delivering to a native valve annulus (e.g., a native mitral valve annulus) of a heart a prosthetic heart valve having a body expandable from a collapsed, delivery configuration to an expanded, deployed configuration. The method can further include, after the delivering, causing the prosthetic heart valve to move from the delivery configuration to the deployed configuration. With the prosthetic heart valve in its deployed configuration, an anchoring tether extending from the prosthetic heart valve can be secured to a wall of the heart. An electrode coupled to at least one of the prosthetic heart valve or the anchoring tether can then be used to at least one of pace the heart or sense a signal associated with the heart.

U.S. Pat. No. 7,643,879 to Shuros et al. describes systems and methods using a heart valve and an implantable medical device, such as for event detection and optimization of cardiac output. The cardiac management system includes a heart valve, having a physiological sensor. The physiological sensor is adapted to measure at least one of an intrinsic electrical cardiac parameter, a hemodynamic parameter or the like. The system further includes an implantable electronics unit, such as a cardiac rhythm management unit, coupled to the physiological sensor of the heart valve to receive physiological information. The electronics unit is adapted to use the received physiological information to control delivery of an electrical output to the subject.

### SUMMARY OF THE APPLICATION

Some embodiments of the present invention provide a prosthetic cardiac valve, which is configured to be implanted in a native cardiac valve of a patient, and which comprises a plurality of prosthetic leaflets, a frame, and one or more electrodes, including a cathode and an anode, mechanically coupled to the frame. The prosthetic cardiac valve further comprises a prosthetic-valve coil, which is in non-wireless electrical communication with the cathode and the anode.

For some applications, the prosthetic cardiac valve further comprises circuitry, which is configured to apply pacing to the heart and/or sense cardiac electrical activity using the one or more electrodes.

In some applications, the prosthetic cardiac valve is configured to be delivered to the native cardiac valve in a constrained delivery configuration, The prosthetic cardiac valve comprises:
a frame, which comprises interconnected stent cells, which include distal stent cells that are located in a distal half of the frame and are shaped so as to define respective distal peaks;
a plurality of prosthetic leaflets coupled to the frame so as to allow blood flow in a downstream direction and inhibit blood flow in an upstream direction when the prosthetic cardiac valve is in an expanded deployment configuration;
electrodes, which include a plurality of distal electrodes mechanically coupled to the frame at or near respective ones of the distal peaks; and
circuitry, which is electrically coupled to the electrodes, and which is configured to apply pacing to the heart using one or more of the distal electrodes as one or more anodes and one or more of the other distal electrodes as one or more cathodes.

There is therefore provided, in accordance with an application of the present invention, a prosthetic cardiac valve system including a prosthetic cardiac valve, which is configured to be delivered to a native cardiac valve of a heart of a patient in a constrained delivery configuration, and which includes:
a frame, which includes interconnected stent cells, which include distal stent cells that are located in a distal half of the frame and are shaped so as to define respective distal peaks;
a plurality of prosthetic leaflets coupled to the frame so as to allow blood flow in a downstream direction and inhibit blood flow in an upstream direction when the prosthetic cardiac valve is in an expanded deployment configuration;
electrodes, which include a plurality of distal electrodes mechanically coupled to the frame at or near respective ones of the distal peaks; and
circuitry, which is electrically coupled to the electrodes, and which is configured to apply pacing to the heart using one or more of the distal electrodes as one or more anodes and one or more of the other distal electrodes as one or more cathodes.

For some applications, the circuitry is configured to apply ventricular pacing to the heart using the one or more of the distal electrodes as the one or more anodes and the one or more of the other distal electrodes as the one or more cathodes.

For some applications, the circuitry is configured to apply atrial pacing to the heart using the one or more of the distal electrodes as the one or more anodes and the one or more of the other distal electrodes as the one or more cathodes.

For some applications, the distal electrodes are disposed at a common axial position along a central longitudinal axis of the frame when the prosthetic cardiac valve is in the expanded deployment configuration.

For some applications, the circuitry is configured to apply the pacing to the heart using exactly one of the distal electrodes as an anode and exactly one of the other distal electrodes as a cathode.

For some applications, the circuitry is configured to apply the pacing to the heart using the one or more of the distal electrodes as the one or more anodes and two or more of the other distal electrodes as two or more cathodes.

For some applications, the circuitry is configured to apply the pacing to the heart using exactly one of the distal electrodes as an anode and the two or more of the other distal electrodes as the two or more cathodes.

For some applications, the distal electrodes are mechanically coupled to the frame at or within 8 mm of the respective ones of the distal peaks.

For some applications, the distal electrodes are mechanically coupled to the frame at the respective ones of the distal peaks.

For some applications, the distal electrodes are mechanically coupled to the frame at or near respective distal peaks of distal-most ones of the distal stent cells.

For some applications, the prosthetic cardiac valve is a prosthetic aortic valve.

For some applications, the distal peaks are respective upstream distal peaks, and the distal electrodes are upstream distal electrodes that are mechanically coupled to the frame at or near respective ones of the upstream distal peaks.

For some applications, the prosthetic cardiac valve is a prosthetic atrioventricular valve.

For some applications, the distal peaks are respective downstream distal peaks, and the distal electrodes are downstream distal electrodes that are mechanically coupled to the frame at or near respective ones of the downstream distal peaks.

There is further provided, in accordance with an application of the present invention, a prosthetic cardiac valve system including a prosthetic cardiac valve, which is configured to be delivered to a native cardiac valve of a heart of a patient in a constrained delivery configuration, and which includes:
a frame, which includes interconnected stent cells, which include distal stent cells that are located in a distal half of the frame and are shaped so as to define respective distal peaks;
a plurality of prosthetic leaflets coupled to the frame so as to allow blood flow in a downstream direction and inhibit blood flow in an upstream direction when the prosthetic cardiac valve is in an expanded deployment configuration;
electrodes, which include a plurality of distal electrodes mechanically coupled to the frame at or near respective ones of the distal peaks; and
circuitry, which is electrically coupled to the electrodes, and which is configured to sense cardiac electrical activity of the patient using one or more of the distal electrodes as one or more anodes and one or more of the other distal electrodes as one or more cathodes.

For some applications, the circuitry is configured to sense an intracardiac electrogram (EGM) of the heart using the one or more of the distal electrodes as the one or more anodes and the one or more of the other distal electrodes as the one or more cathodes.

For some applications, the distal electrodes are disposed at a common axial position along a central longitudinal axis of the frame when the prosthetic cardiac valve is in the expanded deployment configuration.

For some applications, the circuitry is configured to sense the cardiac electrical activity using exactly one of the distal electrodes as an anode and exactly one of the other distal electrodes as a cathode.

For some applications, the distal electrodes are mechanically coupled to the frame at or within 8 mm of the respective ones of the distal peaks.

For some applications, the distal electrodes are mechanically coupled to the frame at the respective ones of the distal peaks.

For some applications, the distal electrodes are mechanically coupled to the frame at or near respective distal peaks of distal-most ones of the distal stent cells.

For some applications, the prosthetic cardiac valve is a prosthetic aortic valve.

For some applications, the distal peaks are respective upstream distal peaks, and the distal electrodes are upstream distal electrodes that are mechanically coupled to the frame at or near respective ones of the upstream distal peaks.

For some applications, the prosthetic cardiac valve is a prosthetic atrioventricular valve.

For some applications, the distal peaks are respective downstream distal peaks, and the distal electrodes are downstream distal electrodes that are mechanically coupled to the frame at or near respective ones of the downstream distal peaks.

The present invention will be more fully understood from the following detailed description of embodiments thereof, taken together with the drawings, in which:

### BRIEF DESCRIPTION OF THE DRAWINGS

FIGS. 1A and 1B are schematic illustrations a prosthetic aortic valve, in accordance with an application of the present invention;

FIG. 2 is a schematic illustration of a valve prosthesis system and the prosthetic aortic valve of FIGS. 1A-B implanted in a body of a patient, in accordance with an application of the present invention;

FIGS. 3A-C are schematic illustrations of a printed circuit board (PCB), an electrical lead, and electrodes, in accordance with an application of the present invention;

FIGS. 3D and 3E are schematic illustrations of a portion of the prosthetic aortic valve of FIGS. 1A-B and the PCB of FIGS. 3A-C coupled to stent struts of the prosthetic aortic valve, in accordance with respective applications of the present invention;

FIGS. 3F and 3G are schematic illustrations additional configurations of the PCB of FIGS. 3A-C, in accordance with respective applications of the present invention;

FIG. 3H is a schematic illustration of another configuration of a frame of the prosthetic aortic valve of FIGS. 1A-B and the PCB of FIGS. 3A-C coupled to the frame, in accordance with an application of the present invention;

FIG. 3I is a schematic illustration of yet another configuration of the PCB of FIGS. 3A-C, in accordance with an application of the present invention;

FIG. 4 is a schematic illustration of a prosthetic atrioventricular valve, in accordance with an application of the present invention;

FIG. 5 is a schematic illustration of an external control unit of a valve prosthesis system comprising the prosthetic aortic valve of FIGS. 1A-B, in accordance with an application of the present invention;

FIGS. 6A-C are schematic cross-sectional illustrations of a native aortic valve from above, indicating angular locations and segments at which pacing pulses may be delivered to the heart, in accordance with respective applications of the present invention;

FIG. 6D is a schematic cross-sectional illustration of the native aortic valve from above, indicating angular locations and segments at which electrical activity of the heart may be sensed, in accordance with respective applications of the present invention;

FIGS. 7A and 7B are a schematic cross-sectional view of the heart from above and a schematic side-view in which a portion of the heart and the native aortic valve have been nearly laid open, indicating vertical locations at which pacing pulses may be delivered to the heart, in accordance with respective applications of the present invention;

FIGS. 8 and 9A-D are schematic illustrations showing results of seven experiments conducted by the inventors, using techniques similar to those described herein;

FIGS. 10A-B are schematic illustrations showing results of another experiment conducted by the inventors, using techniques similar to some of those described herein; and

FIGS. 11A-E are schematic illustrations showing results of yet another experiment conducted by the inventors, using techniques similar to some of those described herein.

### DETAILED DESCRIPTION OF APPLICATIONS

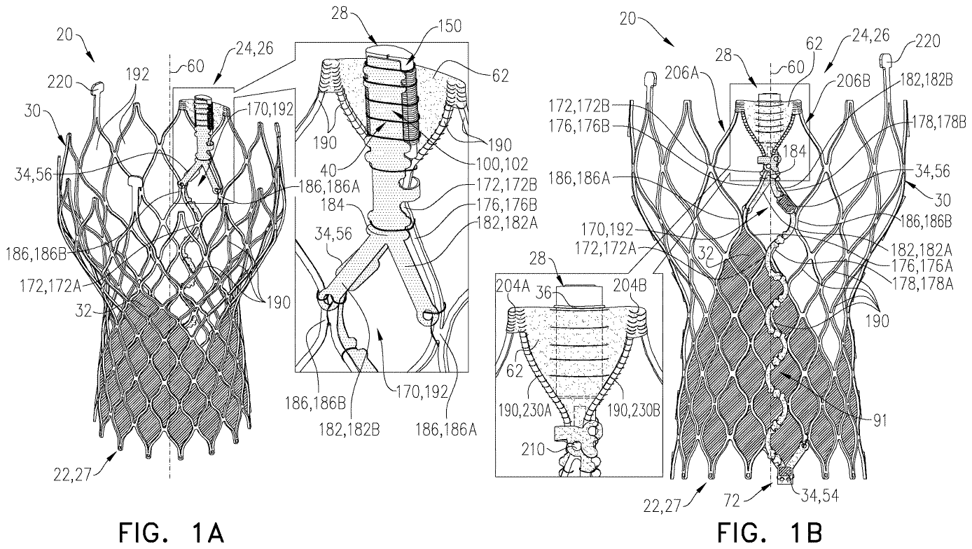

Reference is made to FIGS. 1A and 1B, which are schematic illustrations of a prosthetic aortic valve 20, in accordance with an application of the present invention. For clarity of illustration, only the closer half of prosthetic aortic valve 20 is shown in FIG. 1B.

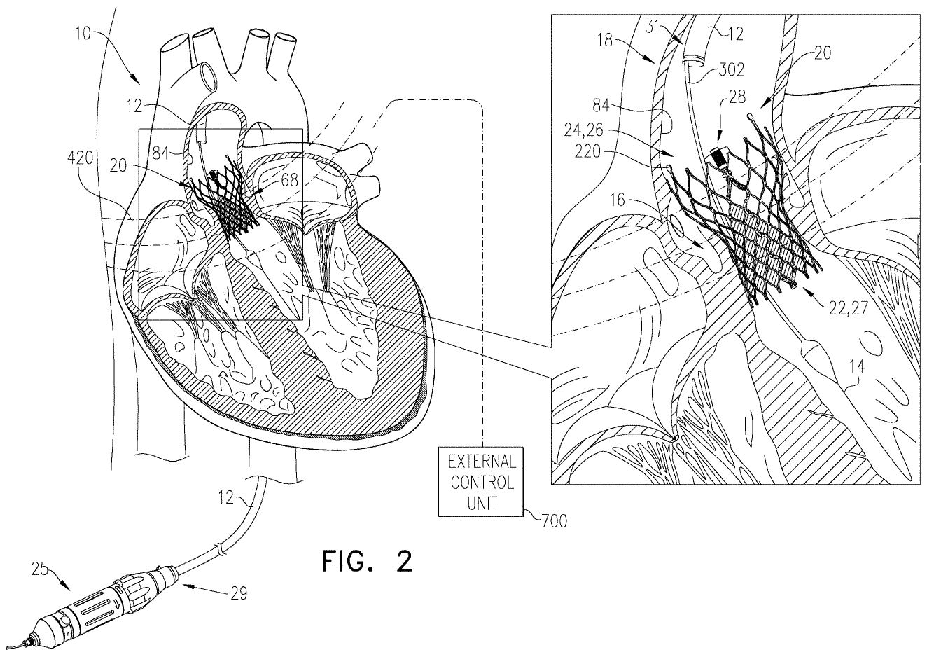

Reference is also made to FIG. 2, which is a schematic illustration of a valve prosthesis system 10 and prosthetic aortic valve 20 implanted in a body of a patient, in accordance with an application of the present invention. Valve prosthesis system 10 further comprises a delivery system 18, which typically comprises a delivery sheath 12 and is used with a guidewire 14. Delivery system 18 typically further comprises a user-control handle 25, which is disposed at (and optionally coupled to) a proximal end portion 29 of delivery sheath 12. The opposite, free end portion of delivery sheath 12 is thus a distal end portion 31 of delivery sheath 12. Prosthetic aortic valve 20 is typically configured to be delivered to a native aortic valve 16 of the patient (or a previously implanted prosthetic aortic valve for TAVI-in-TAV) in a constrained delivery configuration within delivery sheath 12. Distal end portion 31 of delivery sheath 12 may be a conventional tube, for example as shown. Alternatively, distal end portion 31 of delivery sheath 12 may further comprise a capsule that is moveable distally with respect to the remainder of delivery sheath 12 during deployment. All or a portion of prosthetic aortic valve 20 may be contained within the capsule. As used in the present application, including in the claims, in configurations in which distal end portion 31 comprises a capsule (or other type of holder), the distal end portion 31 of delivery sheath 12 refers to the combination of the conventional tubular portion of the sheath and the capsule. By way of example and not limitation, such a capsule is described in U.S. Pat. No. 10,888,421 to Hariton et al., which is incorporated herein by reference.

Typically, prosthetic aortic valve 20 is deployed using imaging, such as fluoroscopy. Optionally, prosthetic aortic valve 20 is rotated and/or axially moved if necessary during the deployment in order to achieve the desired location(s) of at least one of the one or more electrodes 34, described hereinbelow.

Prosthetic aortic valve 20 is shown in FIGS. 1A-B and FIG. 2 in an expanded configuration. Frame 30 defines a central longitudinal axis 60 when prosthetic aortic valve 20 is in this expanded deployment configuration. Prosthetic aortic valve 20 has an upstream end 22 and a downstream end 24. Downstream end 24 may also be a proximal end 26 and upstream end 22 may also be a distal end 27, for example because proximal end 26 may be disposed in distal end portion 31 of delivery sheath 12 more proximally than distal end 27; in other words, proximal end 26 is closer to proximal end portion 29 of delivery sheath 12 than is distal end 27. For some applications, such as shown, proximal end 26 is configured to be coupled to delivery system 18 (e.g., shaped so as to define delivery-tool-coupling tabs 220, which are configured to removably couple frame 30, and thus prosthetic aortic valve 20, to delivery system 18, e.g., to a delivery shaft of delivery system 18, such as described hereinbelow). Typically, as shown in the figures, in configurations in which frame 30 is shaped to define interconnected stent cells 192, delivery-tool-coupling tabs 220 extend axially beyond the interconnected stent cells. For other applications (configuration not shown), distal end 27 is configured to be coupled to delivery system 18, such as to a capsule of the distal end portion 31 of delivery sheath 12, such as described hereinabove.

Prosthetic aortic valve 20 comprises:
a frame 30;
a plurality of prosthetic leaflets 32 coupled to frame 30 so as to allow blood flow in a downstream direction and inhibit blood flow in an upstream direction when prosthetic aortic valve 20 is in the expanded deployment configuration, such as shown in FIGS. 1A-B and 2;
an antenna 28, which is mechanically coupled to frame 30, and which comprises one or more prosthetic-valve coils 36;
one or more electrodes 34, such as cathode 54 and an anode 56, coupled to frame 30; and
optionally, circuitry 40, which is electrically coupled to cathode 54, anode 56, and the one or more prosthetic-valve coils 36.

For some applications, each of electrodes 34 has a conduction surface area of at least 2 mm2, such as at least 2.5 mm2, no more than 20 m2, and/or 2-20 mm2, such as 2.5-20 mm2.

For some applications, a conduction surface of each of electrodes 34 is flat or patterned.

For some applications, a conduction surface of each of electrodes 34 comprises microscopic needles, which may increase the contact with the tissue and promote reendothelialization.

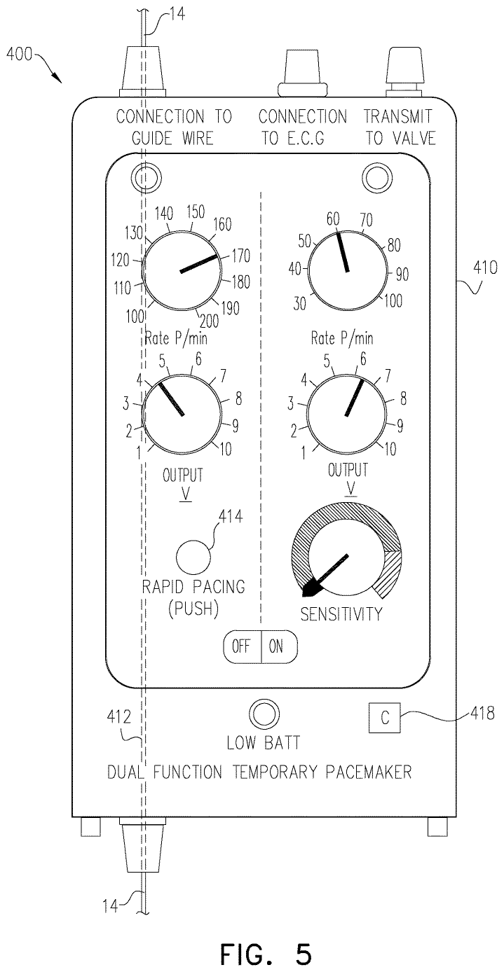

Typically, circuitry 40 is configured to apply pacing to the heart using the one or more electrodes 34. For example, the pacing may be applied temporarily for up to several weeks after implantation of prosthetic aortic valve 20 (e.g., up to one month after implantation), typically using an external control unit to continuously provide power, such as external control unit 400, described hereinbelow with reference to FIG. 5.

Alternatively, for some applications, the pacing is applied longer-term, in which case valve prosthesis system 10 may comprise an energy storage module, e.g., comprising a battery. For example, prosthetic aortic valve 20 may further comprise the energy storage module, e.g., comprising a battery, which may be periodically charged using the external control unit, which may obviate the need for the patient to constantly wear an external energy transmitter. Alternatively or additionally, for example, valve prosthesis system 10 may comprise an implantable energy storage module, e.g., comprising a battery (e.g., a rechargeable battery); for example, the energy storage module may be implantable subcutaneously. The implantable energy storage unit may provide power to prosthetic aortic valve 20 either wirelessly and/or wiredly. For example, the pacing may comprise ongoing sensing of a native electrical signal of the heart and deliverance of electrical stimulus in cases in which the native signal is unsatisfactory for timely ventricular contraction (“VVI pacing”).

Further alternatively or additionally, for some applications, circuitry 40 is configured to apply rapid pacing during an invasive structural heart procedure, such as an implantation procedure, such as a TAVR-in-TAVR procedure in which the first TAVR comprises prosthetic aortic valve 20.

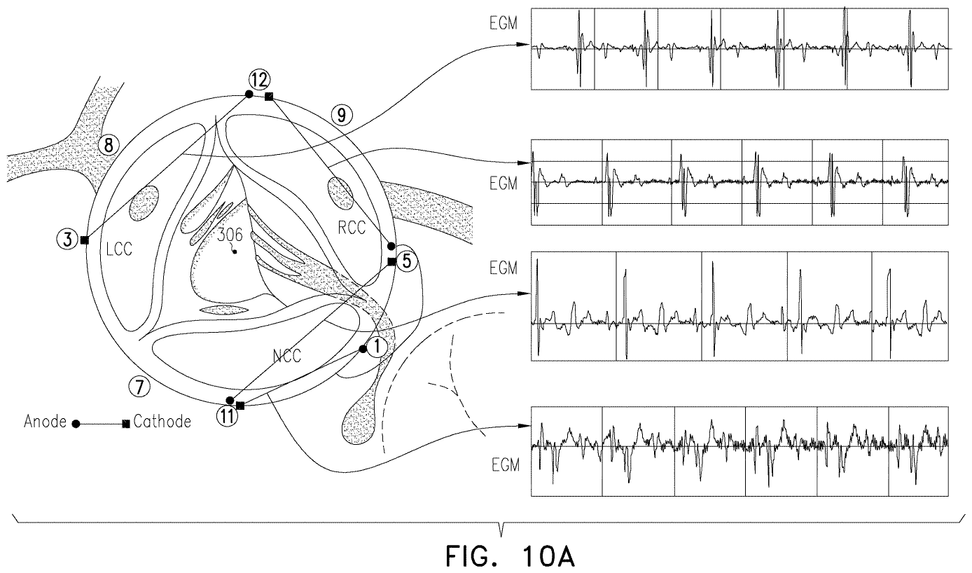

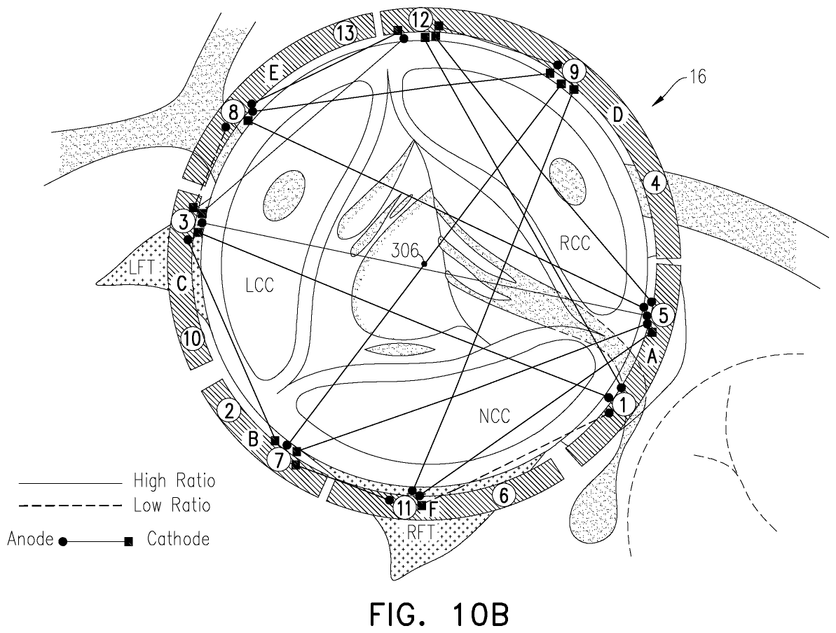

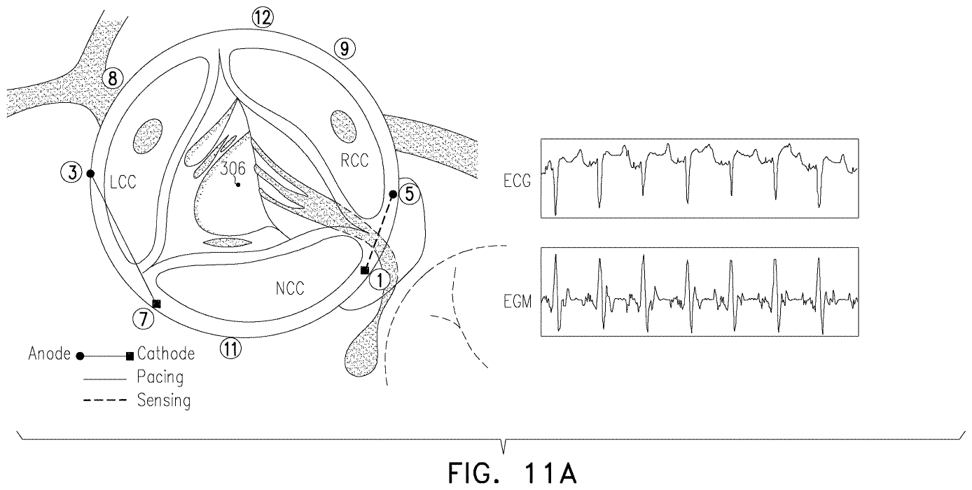

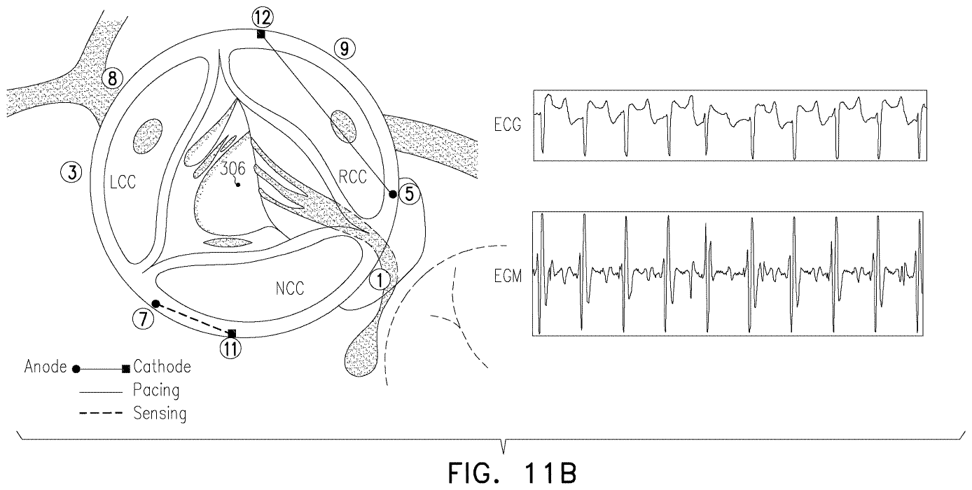

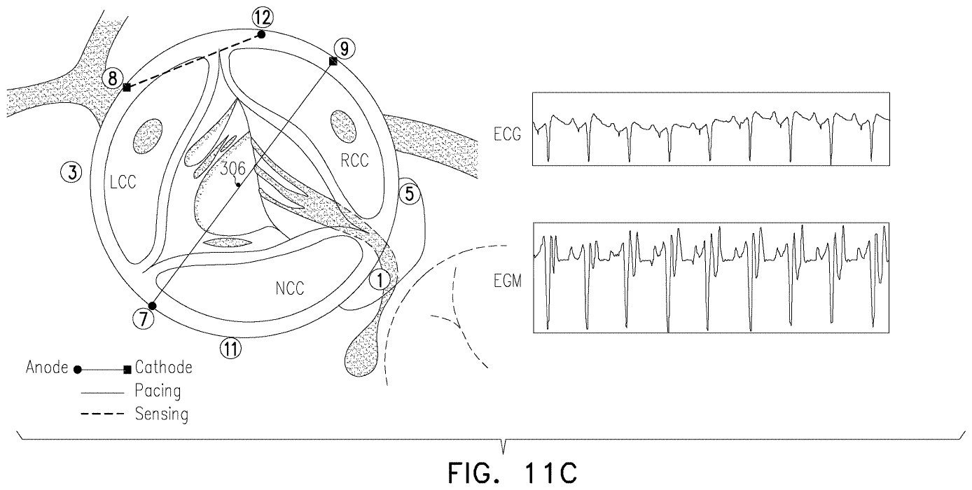

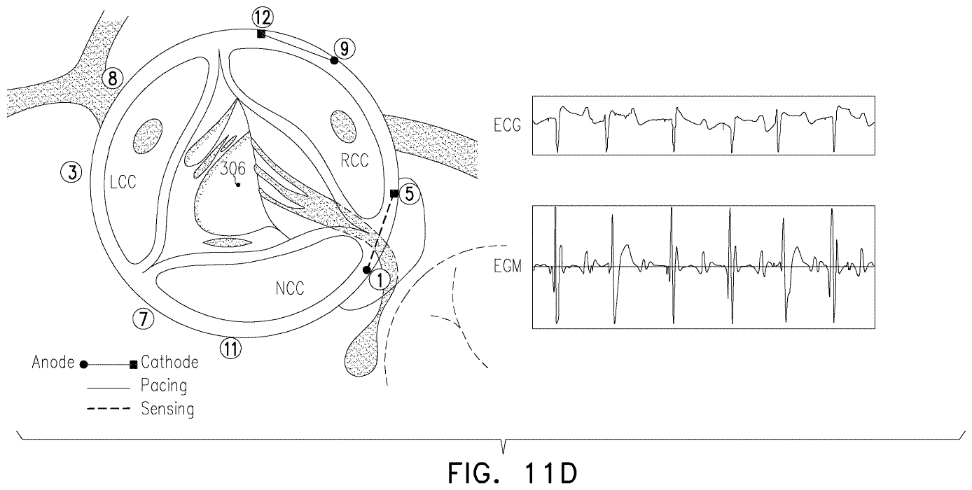

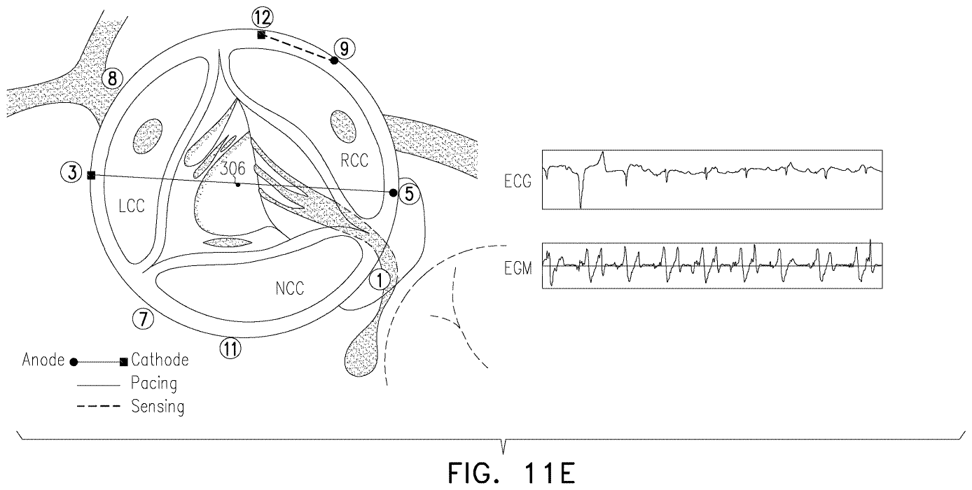

For some applications, prosthetic aortic valve 20 is configured to sense cardiac electrical activity, such as reflected in an electrocardiography (ECG) and/or an intracardiac electrogram (EGM) of the patient's heart. Circuitry 40 may be configured to sense the ECG and/or EGM, or separate circuitry may be provided for sensing the ECG and/or EGM. The ECG and/or EGM sensing may be performed using all or a subset of electrodes 34 and/or one or more separate electrodes may be provided for performing the ECG and/or EGM sensing. Optionally, the EGM is sensed using techniques described hereinbelow with reference to FIGS. 10A-11E.

Each of the one or more prosthetic-valve coils 36 comprises an electrically conductive wire coated with electrical insulation.

Frame 30 typically comprises a stent or other structure, which is typically self-expanding, and may be formed by laser cutting or etching a metal alloy tube comprising, for example, stainless steel or a shape memory material such as Nitinol. For some applications, frame 30 comprises interconnected stent struts 190 arranged so as to define interconnected stent cells 192. Optionally, interconnected stent cells 192 are generally diamond-shaped, such as shown in the drawings.

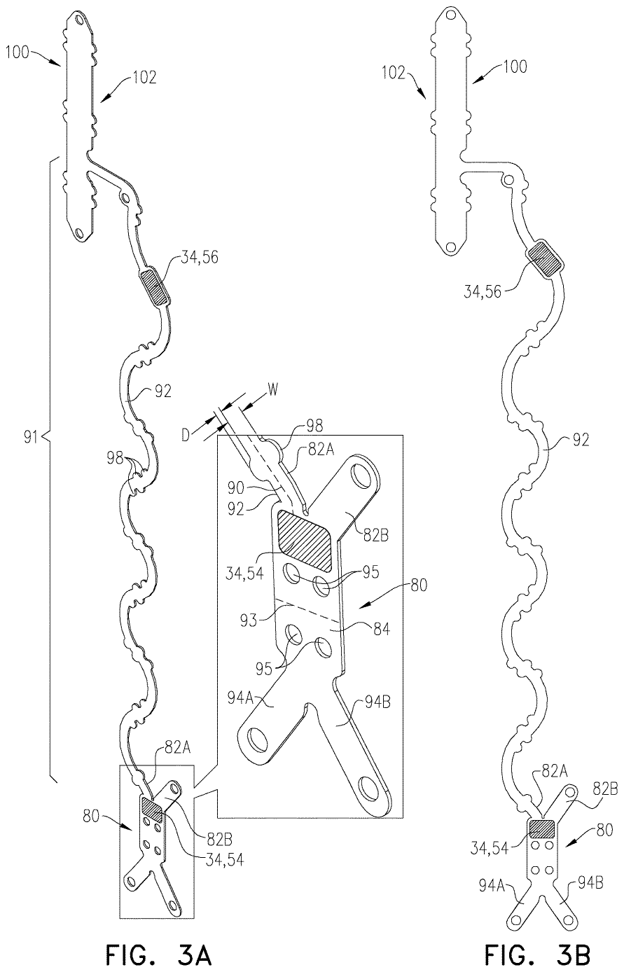

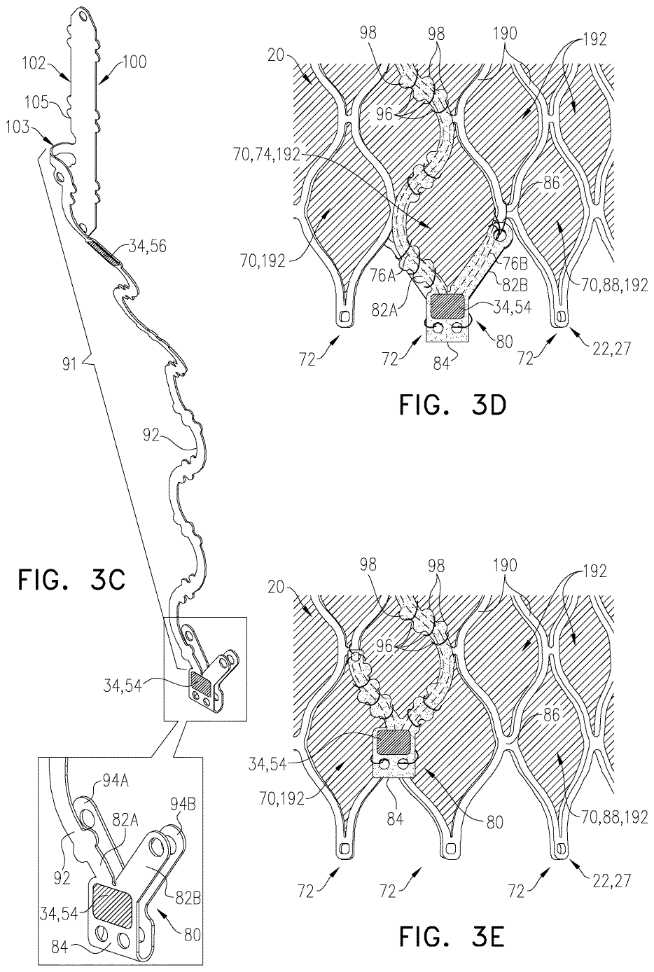

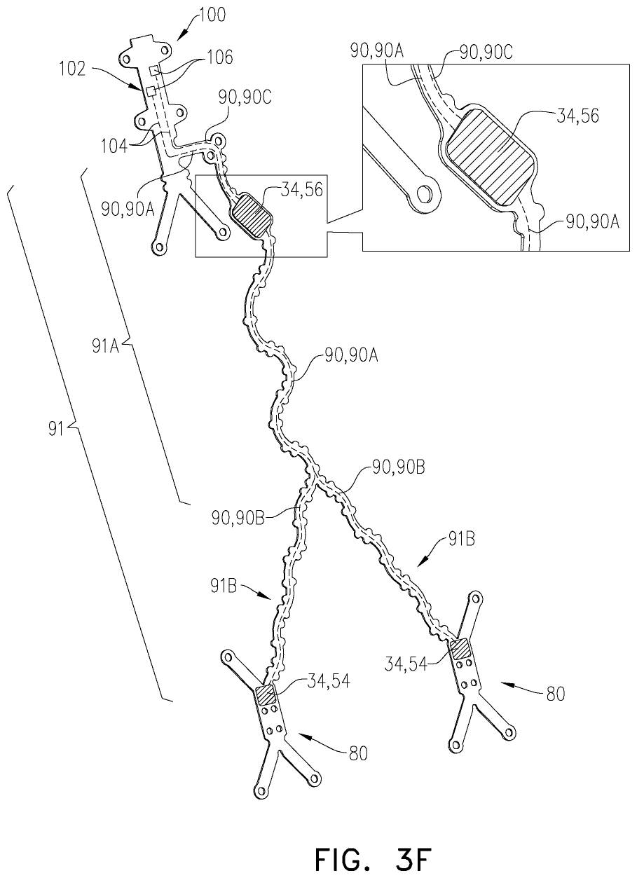

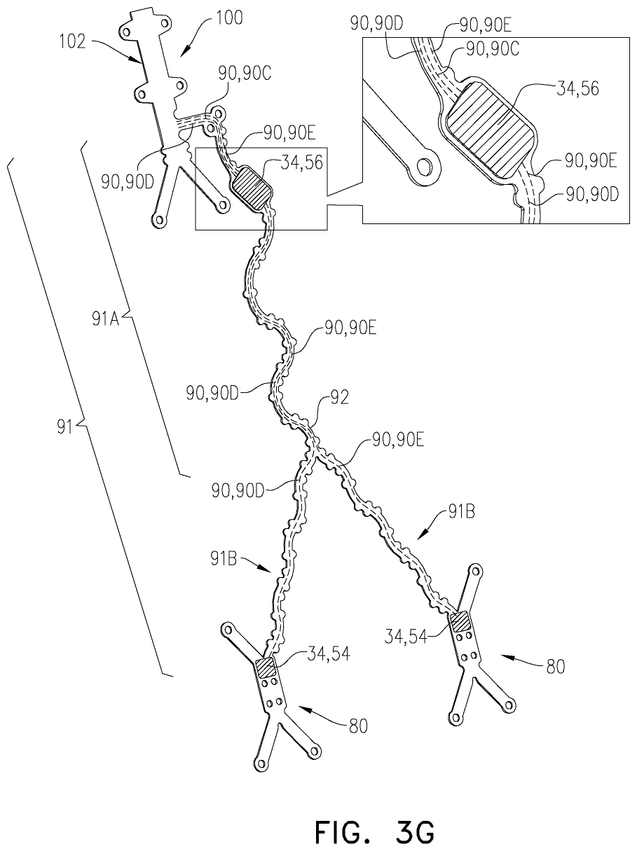

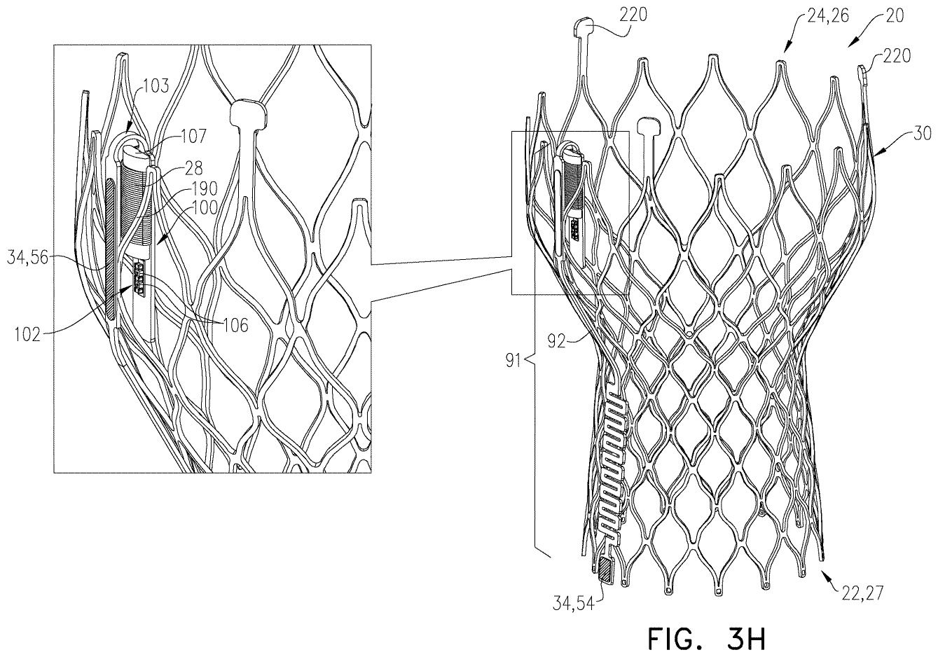

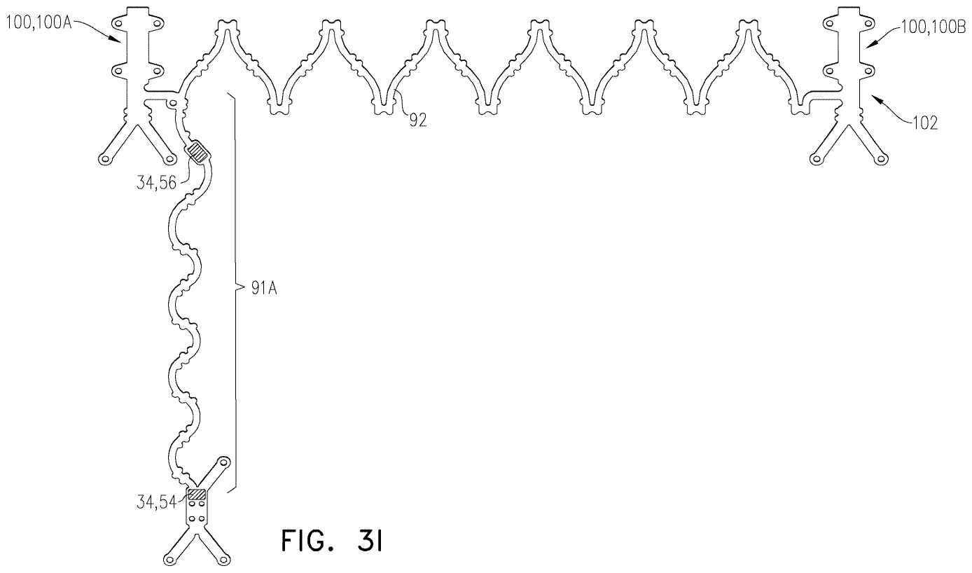

Electrodes 34 may be coupled to frame 30 in various ways. For example, the electrodes may be coupled to stent struts 190 (e.g., by stitching, by soldering, or by using the techniques described hereinbelow with reference to FIGS. 3A-G). Alternatively, the electrodes may be coupled to a skirt or other sheet of thin material that is coupled to stent struts 190, such that the electrodes are coupled to frame 30 via the skirt or other sheet. Further alternatively, the electrodes may be coupled to stent struts 190 such as shown in FIG. 3H, described hereinbelow. Still further alternatively, the electrodes may comprise a coating of conductive material that is coated on a skirt or other sheet of thin material that is coupled to stent struts 190, such that the electrodes are coupled to frame 30 via the skirt or other sheet. Other techniques for coupling the electrodes to frame 30 will be apparent to those skilled in the art who have read the present disclosure, and are within the scope of embodiments of the invention.

Typically, adjoining pairs of prosthetic leaflets 32 are attached to one another at their lateral ends to form commissures, with free edges of the prosthetic leaflets forming coaptation edges that meet one another. Prosthetic leaflets 32 typically comprise a sheet of animal pericardial tissue, such as porcine pericardial tissue, or synthetic or polymeric material. Optionally, prosthetic aortic valve 20 further comprises a skirt. Optionally, leaflets 32 are coupled to frame 30 via being coupled to the skirt, which is coupled to frame 30, or the leaflets are otherwise directly or indirectly coupled to the frame.

For some applications, cathode 54 has a thickness of at least 10 microns, no more than 200 microns, and/or between 10 and 200 microns, e.g., about 50 microns, and/or a surface area of at least 0.5 mm\hat{ }2, e.g., at least 1 mm\hat{ }2; no more than 20 mm\hat{ }2; and/or 0.5-20 mm\hat{ }2, such as 1-20 mm\hat{ }2, in order to provide adequate stimulation. For some applications, cathode 54 is coated with titanium nitride (TiN).

Typically, antenna 28 is mechanically coupled to frame 30 proximal (e.g., downstream) of prosthetic leaflets 32, such as shown. Alternatively, antenna 28 is mechanically coupled to frame 30 distal of prosthetic leaflets 32, or at least partially axially overlapping with prosthetic leaflets 32 (configurations not shown).

Reference is made to FIGS. 1A-B. For some applications:
circumferentially adjacent first and second proximal (e.g., downstream)-most stent cells 206A and 206B of interconnected stent cells 192 are joined at a cell junction 210 (labeled in FIG. 1B),
first proximal (e.g., downstream)-most stent cell 206A comprises a right proximal (e.g., downstream) strut 230A of interconnected stent struts 190, right proximal (e.g., downstream) strut 230A extending between cell junction 210 and a first proximal (e.g., downstream) peak 204A defined by first proximal (e.g., downstream)-most stent cell 206A, and
second proximal (e.g., downstream)-most stent cell 206B comprises a left proximal (e.g., downstream) strut 230B of interconnected stent struts 190, left proximal (e.g., downstream) strut 230B extending between cell junction 210 and a second proximal (e.g., downstream) peak 204B defined by second proximal (e.g., downstream)-most stent cell 206B.

For some applications, prosthetic aortic valve 20 further comprises a flexible sheet 62, which is mechanically coupled to right and left proximal (e.g., downstream) struts 230A and 230B. Optionally, flexible sheet 62 is mechanically coupled to right and left proximal (e.g., downstream) struts 230A and 230B by stitching, such as shown; alternatively or additionally, flexible sheet 62 is mechanically coupled to right and left proximal (e.g., downstream) struts 230A and 230B using alternative coupling techniques that are known in the art.

Flexible sheet 62 may comprise, for example, a polymer (e.g., polyethylene terephthalate (PET) or expanded Polytetrafluoroethylene (ePTFE)) or biological tissue, e.g., a pericardium sheet. Optionally, the material of flexible sheet 62 is woven. Optionally, the material of flexible sheet 62 comprises cloth. Flexible sheet 62 is collapsible with prosthetic aortic valve 20 when loaded into delivery sheath 12.

Antenna 28 is mechanically coupled to frame 30 at least in part by being mechanically coupled to flexible sheet 62 between right and left proximal (e.g., downstream) struts 230A and 230B. Optionally, antenna 28 is mechanically coupled to flexible sheet 62 by stitching, such as shown; alternatively or additionally, antenna 28 is mechanically coupled to flexible sheet 62 using alternative coupling techniques that are known in the art. (Because flexible sheet 62 and antenna 28 are shown from outside prosthetic aortic valve 20 in FIG. 1B, flexible sheet 62 partially obscures the view of antenna 28.)

Optionally, antenna 28 is mechanically coupled to frame 30 at least in part by being mechanically coupled to cell junction 210.

For some applications, flexible sheet 62 has an area of 25-100 mm\hat{ }2.

For some applications, flexible sheet 62 is coupled only to one or more interconnected stent struts 190 of each of first and second proximal (e.g., downstream)-most stent cells 206A and 206B, and not to any interconnected stent struts 190 of other stent cells of frame 30.

For some applications, flexible sheet 62 has three sides.

Typically, flexible sheet 62 is separate and distinct from material of prosthetic leaflets 32.

Reference is now made to FIGS. 3A-C, which are schematic illustrations of a printed circuit board (PCB) 92, an electrical lead 90, and electrodes 34, in accordance with an application of the present invention. PCB 92 typically comprises a polymer, such as polyimide, as is known the PCB art. PCB 92 is typically flexible.

Reference is also made to FIGS. 3D and 3E, which are schematic illustrations of a portion of prosthetic aortic valve 20 and PCB 92 coupled to stent struts 190, in accordance with respective applications of the present invention.

Reference is further made to FIGS. 3F and 3G, which are schematic illustrations additional configurations of PCB 92, in accordance with respective applications of the present invention.

Reference is still further made to FIG. 3H, which is a schematic illustration of frame 30 of prosthetic aortic valve 20 and another configuration of PCB 92 coupled to frame 30, in accordance with an application of the present invention.

Reference is additionally made to FIG. 3I, which is a schematic illustration of yet another configuration of PCB 92, in accordance with an application of the present invention.

FIGS. 3A-C, 3F-G, and 3I show elements of prosthetic aortic valve 20 prior to assembly of prosthetic aortic valve 20, and FIGS. 3D, 3E and 3H show these elements after assembly of prosthetic aortic valve 20. For clarity of illustration, FIG. 3H does not show leaflets 32, although they provided in practice.

In some of the configurations shown in FIGS. 3A-G and 3I (and FIGS. 1A-B), distal (e.g., upstream) ones 70 of interconnected stent cells 192 are located in a distal (e.g., upstream) half of frame 30 (optionally, a distal upstream third, such as a distal upstream quarter of frame 30) and define respective distal (e.g., upstream) peaks 72. At least one electrode 34, such as a cathode 54 (as labeled) or an anode 56 (configuration not labeled), is disposed at or near (e.g., within 8 mm of) a distal (e.g., upstream) peak 72 of one 74 of the distal (e.g., upstream) stent cells 70 (and is thus referred to herein as a distal (e.g., upstream) electrode 34). First and second distal (e.g., upstream) stent struts 76A and 76B of the one 74 of distal (e.g., upstream) stent cells 70 are joined at the distal (e.g., upstream) peak 72 (the distal (e.g., upstream) peak 72 is obscured in FIG. 3D, but can be seen in the adjacent stent cells). Optionally, such as shown, the distal (e.g., upstream) ones 70 of stent cells 192 are distal (e.g., upstream)-most ones of stent cells 192, and the one 74 of distal (e.g., upstream) stent cells 70 is one 74 of distal (e.g., upstream)-most stent cells 192. Alternatively, the distal (e.g., upstream) ones 70 of stent cells 192 are not distal (e.g., upstream)-most ones of stent cells 192, and the one 74 of distal (e.g., upstream) stent cells 70 is not one 74 of distal (e.g., upstream)-most stent cells 192, for example as shown in FIG. 3E.

In some of the configurations shown in FIGS. 3A-G and 3I (and FIGS. 1A-B), at least one electrode 34, such as an anode 56 (as labeled) or a cathode 54 (configuration not labeled), is disposed on a proximal (e.g., downstream) portion of frame 30, such as (a) a proximal (e.g., downstream) half of frame 30, (b) a portion of frame 30 proximal (e.g., downstream) of prosthetic leaflets 32, and/or (c) a portion of frame defined by a proximal-most (e.g., downstream-most) two rows of stent cells. This at least one electrode 34 is thus referred to herein as a proximal (e.g., downstream) electrode 34.

In some of the configurations shown in FIGS. 3A-G and 3I, prosthetic aortic valve 20 further comprises coupling material 80, which is shaped so as to define:
a first strip 82A that is mechanically coupled to first distal (e.g., upstream) stent strut 76A,
a second strip 82B that is mechanically coupled to second distal (e.g., upstream) stent strut 76B, and
a junction 84, which couples together first and second strips 82A and 82B,
such that first and second strips 82A and 82B together couple electrode 34, such as a cathode 54, to frame 30 at or near (e.g., within 8 mm of) distal (e.g., upstream) peak 72. Using first and second strips 82A and 82B in this arrangement to couple electrode 34 to frame 30 typically helps stabilize electrode 34 with respect to frame 30, both during expansion of frame 30 from its compressed elongated state, and during many cardiac cycles after implantation of frame 30.

Optionally, first and second strips 82A and 82B are integrally joined at junction 84, e.g., integrally formed from a single piece of material (such as shown); alternatively, first and second strips 82A and 82B comprise discrete pieces of material coupled together at junction 84 (configuration not shown). First strip 82A may be mechanically coupled to either surface of first distal (e.g., upstream) stent strut 76A, and second strip 82B may be mechanically coupled to either surface of second distal (e.g., upstream) stent strut 76B.

For some applications, first and second strips 82A and 82B are mechanically coupled to first and second distal (e.g., upstream) stent struts 76A and 76B, respectively, by stitching, such as shown (to this end, first and second strips 82A and 82B may comprise stitching holes, as shown).

For some applications, junction 84 of coupling material 80 is mechanically coupled to frame 30 at or near (e.g., within 5 mm of) distal (e.g., upstream) peak 72.

For some applications:
first strip 82A has length equal to at least 50% of a length of first distal (e.g., upstream) stent strut 76A; for example, the length of first strip 82A may be greater than the length of first distal (e.g., upstream) stent strut 76A, such as at least 120% of the length of first distal (e.g., upstream) stent strut 76A (which may aid with mechanically coupling first strip 82A to first distal (e.g., upstream) stent strut 76A), and/or
second strip 82B has length equal to at least 50% of a length of second distal (e.g., upstream) stent strut 76B, such as least 75%, e.g., 100% of the length of second distal (e.g., upstream) stent strut 76B, and/or no more than 100% of the length of second distal (e.g., upstream) stent strut 76B.

For some applications, the one 74 of distal (e.g., upstream) stent cells 70 is a first one 74 of distal (e.g., upstream) stent cells 70, and the first one 74 of distal (e.g., upstream) stent cells 70 is joined at a cell junction 86 (node) to a circumferentially-adjacent second one 88 of distal (e.g., upstream) stent cells 70. Second strip 82B is mechanically coupled to cell junction 86, such as by stitching, such as shown (to this end, second strip 82B may comprise a stitching hole, as shown).

For some applications, prosthetic aortic valve 20 further comprises electrical lead 90 (shown schematically in the enlargement in FIG. 3A), which is electrically coupled to electrode 34 (and typically circuitry 40, if provided). First strip 82A is mechanically coupled to at least a portion of electrical lead 90.

For some of these applications, first strip 82A comprises electrical insulation, and first strip 82A electrically insulates the at least a portion of electrical lead 90 (such that first strip 82A and electrical lead 90 together provide an electrode lead). For some of these applications, first strip 82A comprises an elongate portion 91 of PCB 92 with which electrical lead 90 is integral (e.g., encased within PCB 92, such as by lamination, or disposed on an external surface of PCB 92 and coated with an electrically insulating coating). Typically, electrical lead 90 comprises a track (also known as a conductive trace) of PCB 92. In this configuration, PCB 92 typically also defines second strip 82B and junction 84 of coupling material 80. Although elongate portion 91 of PCB 92 is shown as oriented in a generally distal-proximal (e.g., upstream-downstream) orientation, elongate portion 91 of PCB 92 may also be at least partially oriented in a circumferential (angular) orientation around a portion of frame 30, such as shown in FIG. 3I, in which PCB 92 is shaped so as to define a generally distal-proximal (e.g., upstream-downstream) oriented elongate portion labeled 91, as well as a circumferentially-oriented (angularly-oriented) elongate portion oriented circumferentially (angularly) around a circumferential (angular) portion of frame 30 (horizontal in the figure).

Alternatively, first strip 82A is non-electrically-insulating, in which case electrical lead 90 may be electrically insulated by separate electrical insulation.

For some applications, first and second strips 82A and 82B are outer first and second strips 82A and 82B, which are mechanically coupled to radially outer (with respect to central longitudinal axis 60 of frame 30) sides of first and second distal (e.g., upstream) stent struts 76A and 76B, respectively. Coupling material 80 is shaped so as to further define:
an inner first strip 94A that is mechanically coupled to a radially inner side of first distal (e.g., upstream) stent strut 76A, and
an inner second strip 94B that is mechanically coupled to a radially inner side of second distal (e.g., upstream) stent strut 76B.

Junction 84 of coupling material 80 couples together outer first strip 82A, outer second strip 82B, inner first strip 94A, and inner second strip 94B. Outer first strip 82A, outer second strip 82B, inner first strip 94A, and inner second strip 94B together couple electrode 34 to frame 30 at or near distal (e.g., upstream) peak 72.

For some of these applications, junction 84 of coupling material 80 is folded over distal (e.g., upstream) peak 72, such as shown, such as shown in FIGS. 3C-D. Optionally, the folded junction 84 is mechanically coupled to frame 30 at or near distal (e.g., upstream) peak 72, such as by stitching, such as shown (to this end, junction 84 may comprise stitching holes 95, as shown). Prior to being folded over during assembly of prosthetic aortic valve 20, junction 84 may generally have an X-shape, such as shown in FIGS. 3A-B. A fold line 93 is schematically labeled in the enlargement of FIG. 3A.

Reference is still made to FIGS. 3A-D, and is again made to FIGS. 1A-B. For some applications, electrical lead 90, which electrically couples one or more electrodes 34 to circuitry 40, is integral with elongate portion 91 of PCB 92 (electrical lead 90 is shown schematically in the enlargement of FIG. 3A). As shown in FIGS. 1A-B and 3D, elongate portion 91 of PCB 92 is mechanically coupled to some of interconnected stent struts 190 of frame 30, such as by suturing using sutures 96. Elongate portion 91 of PCB 92 thus serves both to provide electrical insulation to electrical lead 90 and to facilitate coupling of electrical lead 90 to stent struts 190. This encasing of electrical lead 90 in elongate portion 91 of PCB 92 may be implemented either in combination with the techniques for mechanically coupling junction 84 to frame 30 at or near distal (e.g., upstream) peak 72 described above with reference to FIGS. 3A-D, or independently of these techniques.

As described above, for some applications, which electrical lead 90, which electrically couples one or more electrodes 34 to circuitry 40, is integral with elongate portion 91 of PCB 92. For some of these applications, the one or more electrodes 34 are mechanically coupled to frame 30:
downstream of prosthetic leaflets 32 and/or at a proximal (e.g., downstream) half of frame 30, such as shown for anode 56 in FIGS. 1A-B, and/or
upstream of the prosthetic leaflets 32, optionally at or near (e.g., within 8 mm of) respective distal (e.g., upstream) peaks 72 of respective ones 74 of distal (e.g., upstream)-most ones 70 of stent cells 192.

In any of these configurations, the one or more electrodes 34 may be directly coupled to frame 30, or may be indirectly coupled to frame 30 by being coupled to elongate portion 91 of PCB 92, which in turn is directly coupled to frame 30. In addition, in any of these configurations, circuitry 40 may be mechanically coupled to frame 30 proximal (e.g., downstream) of prosthetic leaflets 32.

For some applications, elongate portion 91 of PCB 92 has an undulating shape that generally runs along interconnected stent struts 190, such as shown in FIGS. 1A-B and 3D-E.

Alternatively or additionally, for some applications, elongate portion 91 of PCB 92 is shaped so as to follow a path of interconnected stent struts 190, such as shown in FIGS. 1A-B and 3D-E, and/or has a same general shape as interconnected stent struts 190, also as shown in FIGS. 1A-B and 3D-E.

For some applications, elongate portion 91 of PCB 92 has one or more of the following lengths:
at least 50%, no more than 100%, and/or 50%-100% of a length of frame 30, measured parallel to central longitudinal axis 60 of frame 30 (labeled in FIGS. 1A-B),
at least 150%, no more than 1000%, and/or 150%-1000% of a greatest dimension of circuitry portion 100 of PCB 92, described hereinbelow with reference to FIG. 3F (in configurations in which circuitry portion 100 is elongate, the greatest dimension may equal the length of long lateral side 105 of circuitry portion 100, labeled in FIG. 3C), and/or
at least 0.5 cm, no more than 6 cm, and/or 0.5-6 cm, such as at least 0.5 cm, no more than 4 cm, and/or 0.5-4 cm (e.g., for prosthetic aortic valve 20), or at least 1.5 cm, no more than 6 cm, and/or 1.5-6 cm (e.g., for the mitral or tricuspid valve, such as described hereinbelow with reference to FIG. 5).

All of the above-mentioned lengths of elongate portion 91 are measured in a straight line between endpoints of elongate portion 91, even in configurations in which elongate portion 91 includes curved portions.

Alternatively or additionally, for some applications, a portion of elongate portion 91 between (a) circuitry portion 100 of PCB 92 and (b) a closest of the one or more electrodes 34 coupled by electrical lead 90 to circuitry 40 has one or more of the following lengths:
at least 50%, no more than 100%, and/or 50%-100% of a length of frame 30, measured parallel to central longitudinal axis 60 of frame 30 (labeled in FIGS. 1A-B),
at least 150%, no more than 1000%, and/or 150%-1000% of a greatest dimension of circuitry portion 100 of PCB 92 (in configurations in which circuitry portion 100 is elongate, the greatest dimension may equal the length long lateral side 105 of circuitry portion 100, labeled in FIG. 3C), and/or
at least 0.5 cm, no more than 6 cm, and/or 0.5-6 cm, such as at least 0.5 cm, no more than 4 cm, and/or 0.5-4 cm (e.g., for configurations in which the closest electrode 34 is disposed proximal (e.g., downstream) of prosthetic leaflets 32 and/or at a proximal (e.g., downstream) half of frame 30), or at least 1.5 cm, no more than 6 cm, and/or 1.5-6 cm (e.g., for configurations in which the closest electrode 34 is disposed distal (e.g., upstream) of the prosthetic leaflets 32).

All of the above-mentioned lengths of elongate portion 91 are measured in a straight line between endpoints of elongate portion 91, even in configurations in which elongate portion 91 includes curved portions.

For some applications, elongate portion 91 of PCB 92 has one or more of the following widths (perpendicular to a thickness of PCB 92):
at least 0.4, no more than 1.5, and/or 0.4-1.5 mm, and/or
at least 20%, no more than 120%, and/or 20%-120% of a shortest dimension of circuitry portion 100 of PCB 92 perpendic2ular to a thickness of circuitry portion 100 (in configurations in which circuitry portion 100 is elongate, the shortest dimension may be measured perpendicular to long lateral side 105 of circuitry portion 100, labeled in FIG. 3C).

As mentioned above, electrical lead 90 is coupled to electrode 34. For some applications, electrical lead 90 is coupled to cathode 54, while for other applications, electrical lead 90 is coupled to anode 56. Optionally, more than one electrical lead 90 is integral with elongate portion 91 of PCB 92, in which case a first one of electrical leads 90 may be coupled to cathode 54 and a second one of electrical leads 90 may be coupled to anode 56.

Optionally, a plurality of electrical leads 90 are integral with a corresponding plurality of elongate portions of PCB 92, such as described hereinbelow with reference to FIGS. 3F and 3G.

Optionally, one or more electrodes 34, e.g., one or more cathodes 54 and/or one or more anodes 56, are formed integrally with PCB 92.

Typically, both stent struts 190 and elongate portion 91 of PCB 92 are rectangular in cross section taken perpendicular to respective longitudinal axes of the stent struts and the elongate portion. Typically, electrical lead 90 is also rectangular in cross section, or trapezoidal in cross section. These rectangular cross sections enable flush coupling and/or good crimping of elongate portion 91 to stent struts 190.

For some applications:
stent struts 190 have a thickness of at least 150 microns, such as at least 300 microns; no more than 500 microns; and/or 150-500 microns, such as 300-500 microns,
stent struts 190 have a width of 200-700 microns,
a ratio of the width to the thickness of stent struts 190 is 0.5-2,
electrical lead 90 has a thickness of 5-80 microns, e.g., 50 microns,
electrical lead 90 has a width of 50-300 microns,
a ratio of the width to the thickness of electrical lead 90 is 5-50,
elongate portion 91 of PCB 92 has a thickness of at least 50 microns, no more than 150 microns, and/or 50-150 microns, and/or
elongate portion 91 of PCB 92 has a width of 300-1500 microns, and/or
a ratio of the width to the thickness of elongate portion 91 of PCB 92 is 3-20.

Alternatively or additionally, for some applications:
a ratio of a thickness of stent struts 190 to a thickness of electrical lead 90 is at least 5, no more than 15, and/or 5-15, and/or
a ratio of a thickness of stent struts 190 to a thickness of elongate portion 91 of PCB 92 is at least 2, no more than 5, and/or 2-5.

Elongate portion 91A and/or bifurcation elongate portions 91B of PCB 92, described hereinbelow with reference to FIGS. 3F and/or 3G, may also have the dimensions provided immediately above for elongate portion 91 of PCB 92. Similarly, main portion 90A of electrical lead 90, bifurcation portions 90B of electrical lead 90, electrical lead 90C, electrical lead 90D, and/or electrical lead 90E, described hereinbelow with reference to FIGS. 3F and/or 3G, may also have the dimensions provided immediately above for electrical lead 90.

For some applications, as shown highly schematically in FIG. 3F, PCB 92 comprises a circuitry portion 100, such as an end portion 102 of PCB 92, distinct from elongate portion 91 of PCB 92, and circuitry 40 is coupled to circuitry portion 100 of PCB 92. For some applications, circuitry 40 further comprises (a) tracks 104 (also known as conductive traces) of PCB 92, (b) conductive pads of PCB 92, and (c) electronic components 106 coupled to PCB 92. Elongate portion 91 extends directly from circuitry portion 100 (e.g., end portion 102), and is typically integral with circuitry portion 100 (e.g., end portion 102). (In configurations in which circuitry portion 100 is a mid-portion of PCB 92, rather than end portion 102, PCB 92 extends beyond circuitry portion 100, such as to provide electrical connection to additional elements, e.g., one or more electrodes and/or additional circuitry.) Electrical lead 90 is typically integrally fabricated as a track of elongate portion 91 of PCB 92 in connection with one or more of tracks 104 of PCB 92 that are part of circuitry 40, which obviates the need for a separate connection point between electrical lead 90 and circuitry 40.

For some of these applications, antenna 28 is coupled to circuitry 40 by being coupled to one side of circuitry portion 100 of PCB 92, such as shown in FIGS. 1A-B.

Optionally, elongate portion 91 of PCB 92 is shaped so as to define a plurality of protrusions 98 along elongate portion 91, which inhibit sutures 96 from sliding along elongate portion 91, such that the sutures 96 fix elongate portion 91 of PCB 92 securely to stent struts 190. Typically, protrusions 98 protrude laterally from elongate portion 91 of PCB 92 in a plane defined by PCB 92, either bidirectionally or in a single direction; optionally, some of protrusions 98 protrude bidirectionally and others of protrusions 98 protrude in a single direction, such as shown in the figures. Optionally, as labeled in the enlargement of FIG. 3A, an average distance D of lateral protrusion of protrusions 98 beyond non-protruding portions of elongate portion 91, in a single direction, equals 20%-100% of widths W of elongate portion 91 of PCB 92 at respective locations of the protrusions 98 along elongate portion 91, the average distance D and the widths W measured in the plane defined by PCB 92.

Reference is now made to FIGS. 3F and 3G. In these configurations, elongate portion 91 of PCB 92 is bifurcated, so as to define a main elongate portion 91A and two or more bifurcation elongate portions 91B. Byway of example, exactly two bifurcation elongate portions 91B are shown in FIGS. 3F and 3G; in practice, elongate portion 91 may define more than two bifurcation elongate portions, such as three, four, five, six, or more bifurcation elongate portions.

In some applications, respective electrodes 34, e.g., respective cathodes 54, are coupled to respective bifurcation elongate portions 91B at a respective plurality of angular locations around frame 30. For some applications, such as shown in FIGS. 3F and 3G, respective electrodes 34 (e.g., respective cathodes 54) are disposed at a common axial position along central longitudinal axis 60 of frame 30 when prosthetic aortic valve 20 is in the expanded deployment configuration. For other applications (configuration not shown), respective electrodes 34 are disposed at different respective axial positions along central longitudinal axis 60 when prosthetic aortic valve 20 is in the expanded deployment configuration; for some of these other applications, respective electrodes 34 are disposed at a common angular location with respect to central longitudinal axis 60 when the prosthetic aortic valve is in the expanded deployment configuration.

In some applications, such as shown in FIG. 3F, an electrical lead 90 integral with elongate portion 91 of PCB 92 is bifurcated, so as to define a main portion 90A and two or more bifurcation portions 90B integral with respective bifurcation elongate portions 91B of elongate portion 91 of PCB 92. For example, each of the bifurcation portions 90B of electrical lead 90 may be electrically coupled to a respective electrode 34, e.g., a respective cathode 54, in which case these electrodes are in electrical communication with each other. A separate electrical lead 90C may be provided integral with main elongate portion 91A of elongate portion 91 of PCB 92, in electrical connection with another electrode 34, e.g., an anode 56.

In other applications, such as shown in FIG. 3G, at least two electrical leads 90D and 90E integral with elongate portion 91 of PCB 92. Electrical leads 90D and 90E are partially integral with main elongate portion 91A of elongate portion 91 of PCB 92, and partially integral with respective bifurcation elongate portions 91B of elongate portion 91 of PCB 92. For example, each of electrical leads 90 may be electrically coupled to a respective electrode 34, e.g., a respective cathode 54, in which case these electrodes (e.g., cathodes) are in electrically isolated from each other, and separately electrically connected to circuitry 40. A separate electrical lead 90C may be provided integral with main elongate portion 91A of elongate portion 91 of PCB 92, in electrical connection with another electrode 34, e.g., an anode 56.

For some applications, such as in the configurations described with reference to FIGS. 3F and 3G, circuitry 40 is configured to apply a pacing signal using all of electrodes 34, e.g., all of cathodes 54. For other applications, such as in the configuration described with reference to FIG. 3G, circuitry 40 is configured to apply the pacing signal using fewer than all of electrodes 34, e.g., (a) fewer than all of cathodes 54, for example, using just a single one of cathodes 54, or two or more cathodes 54 of three or more provided cathodes 54, and/or (b) fewer than all of anodes 56, for example, using just a single one of anodes 56, or two or more anodes 56 of three or more provided anodes 56.

Optionally, in configurations in which prosthetic aortic valve 20 comprises a plurality of distal (e.g., upstream) electrodes 34, one or more of the distal (e.g., upstream) electrodes 34 are activated as one or more anodes 56 (e.g., exactly one anode 56), and one or more other distal (e.g., upstream) electrodes 34 are activated as one or more cathodes 54 (e.g., exactly one cathode 54); in other words, any given distal (e.g., upstream) electrode 34 can be activated as either an anode 56 or a cathode 54. In these configurations, circuitry 40 is typically configured to use the one or more anodes 56 and the one or more cathodes 54 for applying pacing and/or for sensing cardiac electrical activity of the patient, such as an intracardiac electrogram (EGM) of the heart. Optionally, the plurality of distal (e.g., upstream) electrodes 34 comprises three or more distal (e.g., upstream) electrodes 34.

For some applications, circuitry 40 is configured to apply the pacing to the heart using the one or more of the distal (e.g., upstream) electrodes 34 as the one or more anodes 56 (e.g., exactly one anode 56) and two or more of the other distal electrodes as two or more cathodes 54. Applying pacing through two or more cathodes 54 simultaneously may improve pacing performance relative to single-cathode pacing, as evidenced by one or more of: a reduction in paced QRS duration, an improvement in hemodynamic response, and/or a decrease in capture threshold. Without being bound by any particular theory, the inventors believe that distributing the cathodal stimulation across a larger effective surface area of the interventricular septum may recruit a broader wavefront of early myocardial activation, thereby reducing total ventricular activation time and improving electromechanical synchrony and therefore hemodynamics.

Alternatively or additionally, optionally, in configurations in prosthetic aortic valve 20 comprises a plurality of proximal (e.g., downstream) electrodes 34, one or more of the proximal (e.g., downstream) electrodes 34 are activated as one or more anodes 56, and one or more other proximal (e.g., downstream) electrodes 34 are activated as one or more cathodes 54; in other words, any given proximal (e.g., downstream) electrode 34 can be activated as either an anode 56 or a cathode 54.

In general, any of electrodes 34 (regardless of their location on frame 30) can be configured as an anode 56 or a cathode 54.

For some applications, circuitry 40 separately activates each of electrodes 34, e.g., cathodes 54 and/or anodes 56, at different times in different combinations, and, based on a determination of which of the electrodes 34 (e.g., cathodes 54, and/or anodes 56 in configurations in which a plurality of anodes 56 are provided) provides the most effective pacing, i.e., the pacing that is successfully obtained using the smallest stimulation voltage. Circuitry 40 uses this most effective combination of electrodes 34, e.g., cathode(s) 54 or anode(s) 56, for future pacing.

For some applications, the determination regarding the most effective pacing is made based on the sensed ECG and/or EGM, as described hereinabove with reference to FIGS. 1A-B and 2, e.g., based on the combination of electrodes that results in the lowest ECG and/or EGM sensing threshold. Alternatively or additionally, for some applications, the determination regarding the most effective pacing is made by selecting the combination of electrodes that yields the lowest power, voltage, or current threshold sufficient for pacing, i.e., successful generation of a cardiac action potential.

In general, circuitry 40 is configured to apply the weakest pacing signal that yields an action potential in the heart. Circuitry 40 may be configured to induce pacing at a set voltage level or alternatively may be set to automatically determine the minimal voltage level of stimulation for a sufficient pacing.

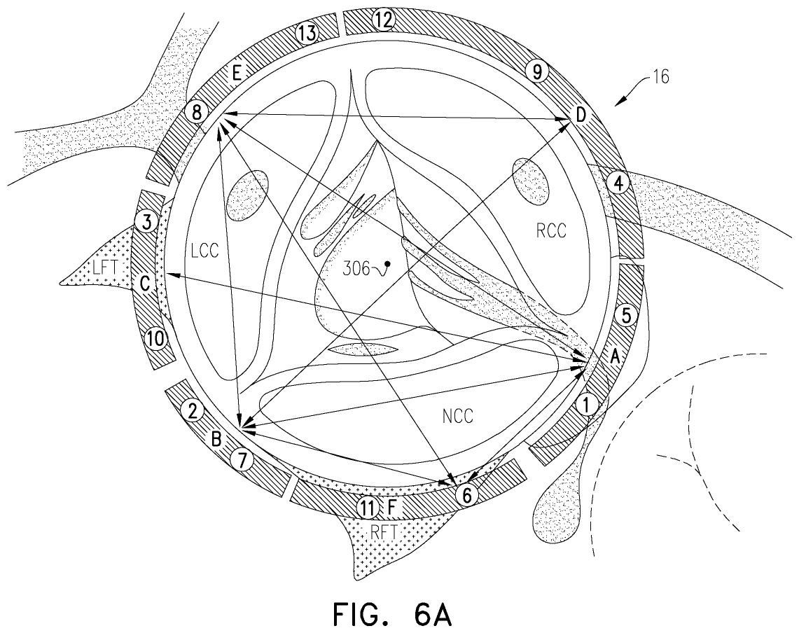

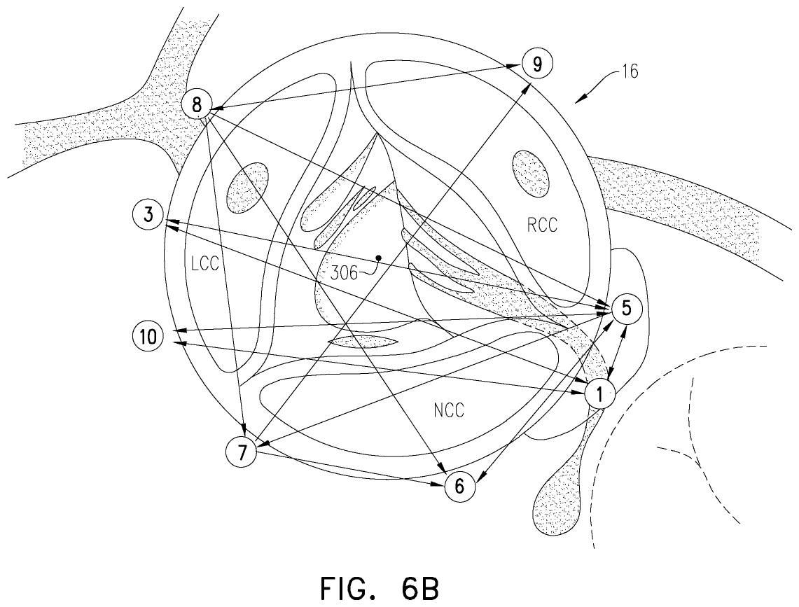

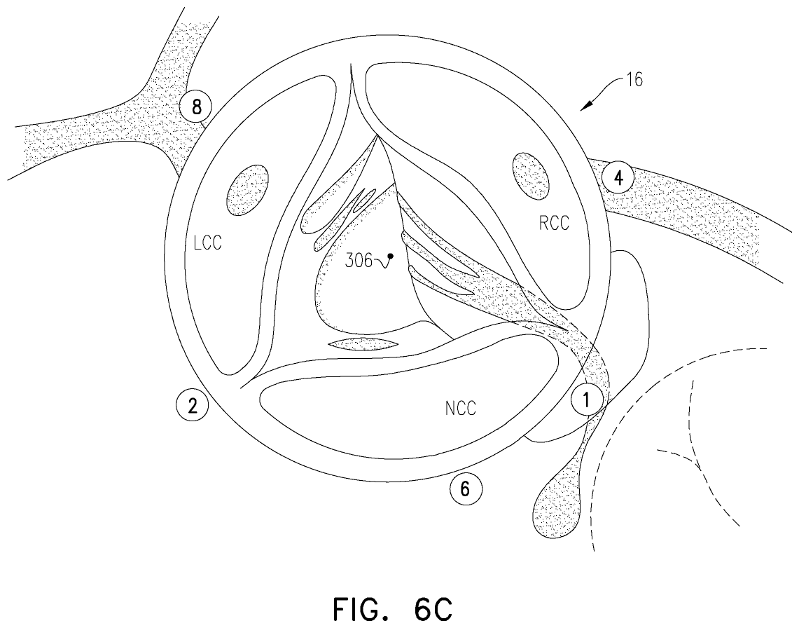

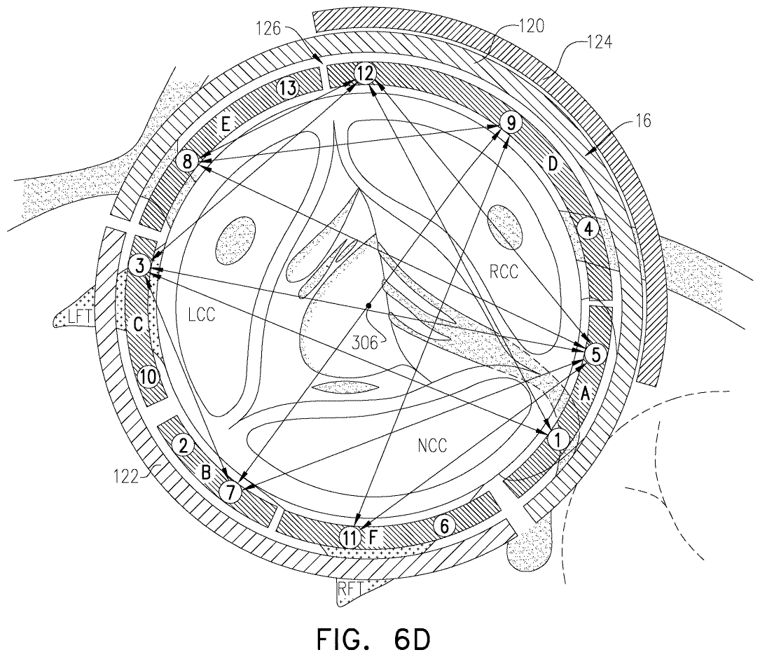

For example, this determination regarding the most effective pacing may be made by circuitry 40 and/or by circuitry of an external control unit, such as external control unit 400, described hereinbelow with reference to FIG. 6. For some applications, this determination is performed (a) only once at the setup of the device immediately after implantation, (b) periodically, e.g., approximately once per day or once per week, and/or (c) before each pacing pulse is applied. An operator may or may not be involved in making the determination.

In some applications, this determination regarding the most effective pacing may be made by activating one or more of the distal (e.g., upstream) electrodes 34 as one or more anodes 56 (rather than as cathodes 54 as labeled in the drawings), and/or activating the distal (e.g., upstream) electrode 34 (or one or more of the distal (e.g., upstream) electrodes if a plurality are provided) as one or more cathodes 54 (rather than as one or more anodes 56 as labeled in the drawings).

Reference is still made to FIGS. 3F-G. It is noted that for clarity of illustration, electrical lead 90 (including main portion 90A and bifurcation portions 90B), electrical lead 90C, electrical lead 90D, and/or electrical lead 90E are shown highly schematically in FIGS. 3F-G. In practice, these electrical leads are typically rectangular in cross section, e.g., having the exemplary dimensions provided hereinabove with reference to FIGS. 1A-B and 3A-D. In addition, these electrical leads may be disposed running alongside one another, such as shown in FIGS. 3F-G, and/or in layers with PCB 92 (configuration not shown), as is known in the PCB art.

Reference is made to FIGS. 3C and 3H. In the configurations shown in these figures, elongate portion 91 extends directly from circuitry portion 100 (e.g., end portion 102), and is typically integral with circuitry portion 100 (e.g., end portion 102). An end portion 103 of elongate portion 91 is bent in a curve over at least a portion of circuitry portion 100, so as to sandwich one or more stent struts 190 between circuitry portion 100 and elongate portion 91, such as shown in FIG. 3H (although not shown in FIG. 3C for the sake of clarity, stent struts 190 are in fact present in prosthetic aortic valve 20). The configurations shown in FIGS. 3C and 3H may optionally be implemented in combination with the other configurations shown herein. (In practice, the one or more stent struts 190 are typically sandwiched more snugly between circuitry portion 100 and elongate portion 91 than shown in FIG. 3H.)

Typically, circuitry portion 100 is disposed radially inward from stent struts 190, end portion 103 of elongate portion 91 is bent in a curve over at least a portion of circuitry portion 100, and the non-curved portion of elongate portion 91 that extends distal (e.g., upstream) from end portion 103 is disposed radially outward from stent struts 190.

For some applications, circuitry portion 100 is elongate, and end portion 103 of elongate portion 91 extends from a long lateral side 105 of circuitry portion 100, such as shown in FIG. 3C, or from a proximal (e.g., downstream) end 107 of circuitry portion 100, such as shown in FIG. 3H. By contrast, if end portion 103 of elongate portion 91 were to instead extend from a distal (e.g., upstream) end of circuitry portion 100, elongate portion 91 might be more likely to be cut during crimping of frame 30. The circumferential width of stent cells 192 diminishes during crimping, while the height of stent cells 192 (in the axial direction) extends during crimping. If the rectangularly cross-sectioned elongate portion 91 were to cross the frame wall when extending from the distal (e.g., upstream) end of circuitry portion 100, elongate portion 91 might be squeezed between two struts during crimping, because the width of elongate portion 91 might be greater than the minimal distance between adjacent nodes or struts during crimping.

Reference is made to FIG. 3I. In this configuration, PCB 92 is shaped so as to define two or more circuitry portions 100 including a first circuitry portion 100A and a second circuitry portion 100B, for example, exactly two circuitry portions 100 (as shown) or three or more circuitry portions 100 (configuration not shown). One or more elongate circuitry-connecting portions 110 of PCB 92 connect the two or more circuitry portions 100. Typically, each of the one or more elongate circuitry-connecting portions 110 comprises one or more electrical leads that are integral with the respective elongate circuitry-connecting portion 110. For some applications, the one or more elongate circuitry-connecting portions 110 extend circumferentially around at least a portion of frame 30.

For some applications, the one or more elongate circuitry-connecting portions 110 are mechanically coupled to some of interconnected stent struts 190 of frame 30, and typically generally run along these stent struts (such that the one or more elongate circuitry-connecting portions 110 may have a zig-zag shape, for example).

Optionally, one of the two or more circuitry portions 100 (e.g., second circuitry portion 100B, as shown) is end portion 102 of PCB 92.

For some applications, circuitry 40 is distributed among the two or more circuitry portions 100, i.e., the two or more circuitry portions 100 comprises respective portions of electronic components of circuitry 40. This may allow the accommodation of circuitry 40 is case a single circuitry portion 100 does not have a sufficient surface area. For some applications, prosthetic aortic valve 20 comprises an energy storage module, e.g., comprising a battery, which is coupled to one of circuitry portions 100.

As used in the present application, including in the claims, “circuitry” means a combination of (a) one or more electronic components 106 and (b) one or more tracks 104 (also known as conductive traces) of a PCB electrically coupled to the one or more electrically components, typically by conductive pads of the PCB. The circuitry may or may not comprise a source of power. The one or more electronic components can be active components (e.g., semiconductor devices, such as integrated circuits, transistors, and/or active diodes); passive components (e.g., electrodes, capacitors, and/or passive diodes); and/or energy storage modules (e.g., comprising a battery). As used in the present application, including in the claims, tracks (also known as traces), electrical leads, wires, and cables are not considered to be electronic components.

Reference is again made to FIGS. 1A-B. The following configuration may be implemented alone or in combination with any of the other configurations described herein, including hereinabove with reference to FIGS. 3A-G and 3I. In this configuration, interconnected stent cells 192 of interconnected stent struts 190 of frame 30 include a first stent cell 170 shaped so as to define:
two peaks 172, consisting of a distal (e.g., upstream) peak 172A and a proximal (e.g., downstream) peak 172B,
two lateral nodes 186, consisting of a left lateral node 186A and a right lateral node 186B,
two left stent struts 176, consisting of (a) a distal (e.g., upstream) left stent strut 176A joined with distal (e.g., upstream) peak 172A and left lateral node 186A, and (b) a proximal (e.g., downstream) left stent strut 176B joined with proximal (e.g., downstream) peak 172B and left lateral node 186A, and
two right stent struts 178, consisting of (a) a distal (e.g., upstream) right stent strut 178A joined with distal (e.g., upstream) peak 172A and right lateral node 186B, and (b) a proximal (e.g., downstream) right stent strut 178B joined with proximal (e.g., downstream) peak 172B and right lateral node 186B.

For example, first stent cell 170 may be located in a proximal (e.g., downstream) half of frame 30, such as shown, e.g., first stent cell 170 may be a proximal (e.g., downstream)-most stent cell (configuration not shown). Alternatively, first stent cell 170 may be located in a distal (e.g., upstream) half of frame 30 (configuration not shown in FIGS. 1A-B), e.g., first stent cell 170 may be a distal (e.g., upstream)-most stent cell (configuration not shown in FIGS. 1A-B).

In this configuration, prosthetic aortic valve 20 comprises an electronic component 150, which is disposed at or near one of peaks 172. For example, electronic component 150 may be part of circuitry 40 (such as shown), may comprise antenna 28 (also such as shown), may comprise an energy storage module, e.g., comprising a battery, or may comprise an electrode 34.

In this configuration, prosthetic aortic valve 20 further comprises coupling material 180, which is shaped so as to define:
a first strip 182A that is mechanically coupled to at least one of left stent struts 176,
a second strip 182B that is mechanically coupled to at least one of right stent struts 178, and
a junction 184, which couples together the first and the second strips 182A and 182B,
such that first and second strips 182A and 182B together couple electronic component 150 to frame 30 at or near (e.g., within 15 mm of) the one of peaks 172. Using first and second strips 182A and 182B in this arrangement to couple electronic component 150 to frame 30 typically helps stabilize electronic component 150 with respect to frame 30, both during expansion of frame 30 from its compressed elongated state, and during many cardiac cycles after implantation of frame 30.

By way of example and not limitation, in FIGS. 1A-B, first strip 182A is shown mechanically coupled to proximal (e.g., downstream) left stent strut 176B, and second strip 182B is shown mechanically coupled to proximal (e.g., downstream) right stent strut 178B, such that first and second strips 182A and 182B together couple electronic component 150 to frame 30 at or near proximal (e.g., downstream) peak 172B. Alternatively, first strip 182A may be mechanically coupled to distal (e.g., upstream) left stent strut 176A, and second strip 182B may be mechanically coupled to distal (e.g., upstream) right stent strut 178A, such that first and second strips 182A and 182B together couple electronic component 150 to frame 30 at or near distal (e.g., upstream) peak 172A (configuration not shown).

Optionally, first and second strips 182A and 182B are integrally joined at junction 184, e.g., integrally formed from a single piece of material (such as shown); alternatively, first and second strips 182A and 182B comprise discrete pieces of material coupled together at junction 184 (configuration not shown). First strip 182A may be mechanically coupled to either surface of the at least one of left stent struts 176, and second strip 182B may be mechanically coupled to either surface of the at least one of right stent struts 178.

For some applications, first and second strips 182A and 182B together couple electronic component 150 to frame 30 at least partially outside the first stent cell at or near the one of peaks 172.

For some applications, first and second strips 182A and 182B are mechanically coupled to the at least one of left stent struts 176 and the at least one of right stent struts 178, respectively, by stitching.

For some applications, junction 184 of coupling material 180 is mechanically coupled to frame 30 at or near the one of peaks 172, such as by stitching.

For some applications, first strip 182A has length equal to at least 50% of a length of the at least one of left stent struts 176; for example, the length of first strip 182A may be greater than the length of the at least one of left stent struts 176. Alternatively or additionally, for some applications, second strip 182B has length equal to at least 50% of a length of the at least one of right stent struts 178; for example, the length of second strip 182B may be greater than the length of the at least one of right stent struts 178.

For some applications, first strip 182A is mechanically coupled to left lateral node 186A, such as by stitching. Alternatively or additionally, for some applications, second strip 182B is mechanically coupled to right lateral node 186B, such as by stitching.

For some applications, prosthetic aortic valve 20 further comprises an electrical lead, such as electrical lead 90, which is electrically coupled to electronic component 150, and first strip 182A is mechanically coupled to at least a portion of the electrical lead. For some of these applications, first strip 182A comprises electrical insulation, and first strip 182A electrically insulates the at least a portion of the electrical lead. For some applications, first strip 182A comprises an elongate portion of a PCB with which the electrical lead is integral, such as elongate portion 91 of PCB 92.

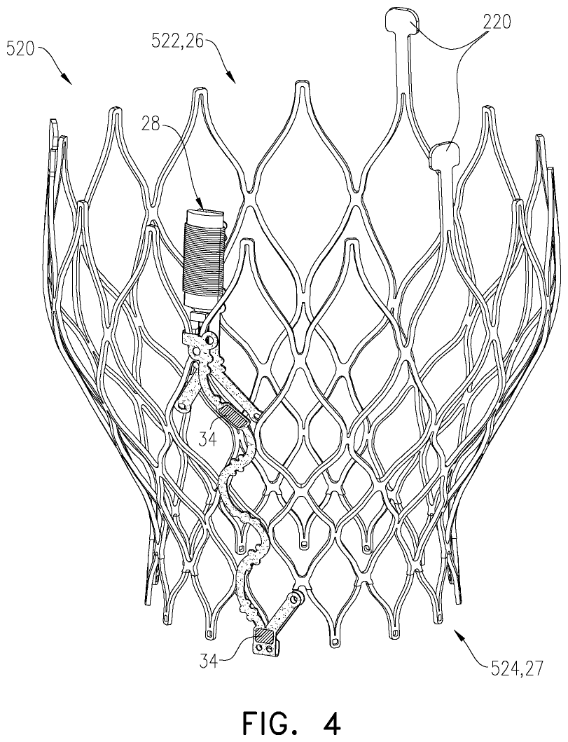

In some applications of the present invention, the prosthetic valves described herein implement some or all of the features described in PCT Publication WO 2025/041129 to Gross et al., with reference to FIGS. 4, 5A-B, 6A-B, 7, 8A-B, and/or 10 thereof, mutatis mutandis.

Reference is now made to FIG. 4, which is a schematic illustration of a prosthetic atrioventricular valve 520, in accordance with an application of the present invention. Other than as described hereinbelow, prosthetic atrioventricular valve 520 may be generally similar to prosthetic aortic valve 20, described hereinabove with reference to FIGS. 1A-3I, and may implement any of the features thereof, mutatis mutandis. Prosthetic atrioventricular valve 520 may also optionally implement any of the features described in PCT Publication WO 2025/041129 to Gross et al., with reference to FIGS. 4-8B thereof, mutatis mutandis.

Prosthetic atrioventricular valve 520 may, for example, be a prosthetic mitral valve or a prosthetic tricuspid valve. Typically, but not necessarily, prosthetic atrioventricular valve 520 has a shorter length than the configurations of prosthetic aortic valve 20 shown in the figures. Optionally, prosthetic atrioventricular valve 520 implements any techniques known in the art for transcatheter prosthetic atrioventricular valves. For example, prosthetic atrioventricular valve 520 may implement techniques described in PCT Publication WO 2022/118316 to Albitov et al., U.S. Pat. No. 11,246,704 to Hariton et al., US Patent Application Publication 2015/0328000 to Ratz et al., PCT Publication WO 2024/010739 to Garete et al., U.S. Pat. No. 10,973,628 to Levi, U.S. Pat. No. 10,299,927 to McLean et al., U.S. Pat. No. 10,828,153 to Noe et al., and/or U.S. Pat. No. 7,510,575 to Spenser et al., all of which are incorporated herein by reference.

Prosthetic atrioventricular valve 520 comprises a frame 530 and a plurality of prosthetic leaflets coupled to frame 530 so as to allow blood flow in a downstream direction and inhibit blood flow in an upstream direction when prosthetic atrioventricular valve 520 is in an expanded deployment configuration, such as shown in FIG. 4. For clarity of illustration, the prosthetic leaflets are not shown in FIG. 4; in practice they are provided, and may be similar to prosthetic leaflets 32, described hereinabove, mutatis mutandis, and/or may implement any techniques of prosthetic leaflets of prosthetic atrioventricular valves known in art, including, but not limited to, the techniques described in the patent publications incorporated hereinabove.

Prosthetic atrioventricular valve 520 has an upstream end 522 and a downstream end 524. Upstream end 522 may also be a proximal end 26 and downstream end 524 may also be a distal end 27, for example because proximal end 26 may be disposed in distal end portion 31 of delivery sheath 12 (described hereinabove with reference to FIG. 2) more proximally than distal end 27; in other words, proximal end 26 is closer to proximal end portion 29 of delivery sheath 12 than is distal end 27. For some applications, such as shown, proximal end 26 is configured to be coupled to delivery system 18 (e.g., shaped so as to define delivery-tool-coupling tabs 220, which are configured to removably couple frame 30, and thus prosthetic atrioventricular valve 520, to delivery system 18, e.g., to a delivery shaft of delivery system 18, such as described herein). For other applications (configuration not shown), distal end 27 is configured to be coupled to delivery system 18, such as to a capsule of the distal end portion 31 of delivery sheath 12, such as described hereinabove.

In some applications of the present invention, prosthetic atrioventricular valve 520 implements any of the techniques described hereinabove with reference to FIGS. 1A-4 for prosthetic aortic valve 20. In some of these techniques, “proximal” features of prosthetic aortic valve 20 are described as “downstream” features, and “distal” features of prosthetic aortic valve 20 are described as “upstream” features. Typically, in prosthetic atrioventricular valve 520 these directions are the opposite, such that “proximal” features of atrioventricular valve 520 are “upstream” features, and “distal” features of atrioventricular valve 520 are “downstream” features.

Reference is now made to FIG. 5, which is a schematic illustration of an external control unit 400 of valve prosthesis system 10, in accordance with an application of the present invention. A prosthetic cardiac valve, such as prosthetic aortic valve 20, prosthetic atrioventricular valve 520, prosthetic atrioventricular valve 720, prosthetic atrioventricular valve 820, or prosthetic atrioventricular valve 920, is configured to be delivered to a native cardiac valve of a patient in a constrained delivery configuration within delivery sheath 12 using guidewire 14, such as described hereinabove with reference to FIG. 2.

External control unit 400 is configured to be disposed outside a body of the patient, and comprises:
a housing 410, which is shaped so as to define a guidewire-receiving channel 412;
a rapid-pacing user control 414; and
external-unit control circuitry 418.

Reference is again made to FIG. 2. Typically, an external system is provided that is configured to be disposed outside a body of the patient. The external system comprises an external control unit 700, which may, for example, comprise external control unit 400, described hereinabove with reference to FIG. 5.

For some applications, the external system further comprises an external transmitter and/or receiver, which optionally comprises an external coil 420, which is highly schematically illustrated in FIG. 2. For example, external coil 420 may be configured to be placed around the patient's chest, such as schematically shown in FIG. 2, or placed against the chest without surrounding the chest, such as against the sternum (configuration not shown). The external transmitter and/or receiver is configured to wirelessly transfer energy to at least one of the one or more prosthetic-valve coils 36, such as by driving external coil 420 to wirelessly transfer the energy to at least one of the one or more prosthetic-valve coils 36 by inductive coupling. For example, the external transmitter may transmit RF energy at a frequency of 1-300 MHz, such as 1-300 MHz, e.g., 6.78 MHz.

Reference is again made to FIG. 5. As described hereinabove with reference to FIGS. 1A-B and 2, for some applications, circuitry 40 is configured to apply both regular pacing and rapid pacing. For example, the rapid pacing may be applied during an invasive structural heart procedure, such as an implantation procedure, such as a TAVR-in-TAVR procedure in which the first TAVR comprises prosthetic aortic valve 20, and a portion of the regular pacing may be applied temporarily while the patient is hospitalized after implantation of prosthetic aortic valve 20. External control unit 400 may be provided for controlling both the regular pacing and the rapid pacing. (When the patient is discharged from the hospital, an external control unit is typically provided having fewer or no user controls accessible by the patient.) Because the user or healthcare works may have access to external control unit 400, it is desirable to prevent accidental activation of rapid pacing after completion of the implantation procedure.

For some applications, external-unit control circuitry 418 is configured to:
drive an external transmitter to wirelessly transfer energy to at least one of the one or more prosthetic-valve coils 36, such as for powering regular pacing (for example, by driving an energy-transmission coil of the external transmitter to wirelessly transfer the energy by inductive coupling), and
only upon activation of rapid-pacing user control 414 and when guidewire 14 is disposed within guidewire-receiving channel 412 of housing 410, drive the prosthetic aortic valve to apply rapid pacing using cathode 54 and anode 56.

To this end, external control unit 400 comprises a sensor, configured to sense whether guidewire 14 is disposed within guidewire-receiving channel 412 of housing 410.

This feature may serve as a safety feature, which restricts application of the rapid pacing to a transcatheter or surgical cardiovascular operation by a certified medical interventionalist.

For some applications, external-unit control circuitry 418 is configured to drive the one or more electrodes 34 to apply pacing; in this configuration, circuitry 40 of prosthetic aortic valve 20, if even provided, is generally passive, i.e., external-unit control circuitry 418 sets the parameters of the pacing signal. For example, in this configuration, circuitry 40 may comprise only passive electrical components, e.g., electrodes, capacitors, and/or passive diodes. Optionally, these techniques are implemented in combination with passive circuitry techniques described in U.S. Pat. No. 11,291,844 to Gross and/or PCT Publication WO 2022/149130 to Gross, both of which are incorporated herein by reference.

Reference is now made to FIGS. 6A-C, which are schematic cross-sectional illustrations of native aortic valve 16 from above, indicating angular locations and segments at which pacing pulses may be delivered to a heart, in accordance with respective applications of the present invention. In FIG. 6A, as well as FIGS. 6D and 10B, described hereinbelow, small gaps are shown between the angular segments for clarity of illustration only; in actuality, adjacent angular segments are contiguous with one another, i.e., are not separated by gaps.

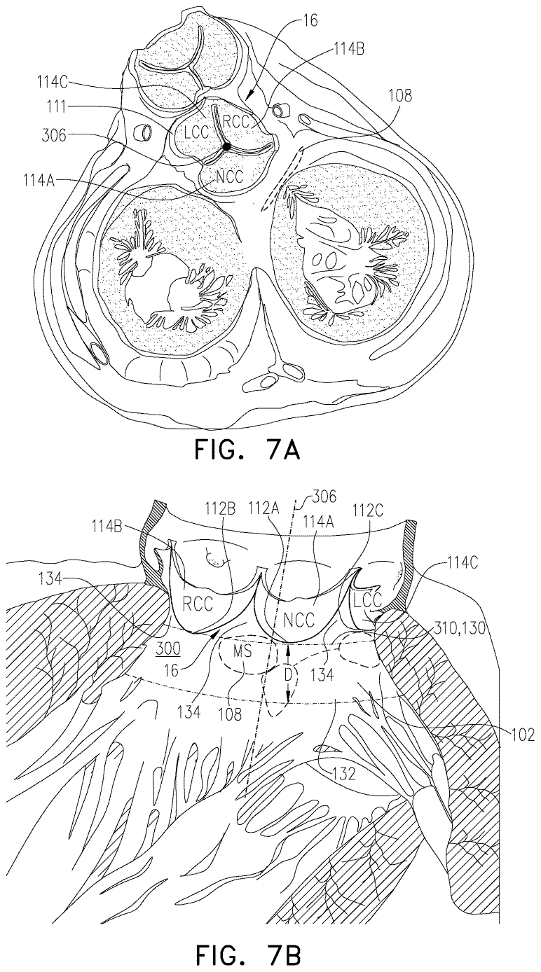

Reference is also made to FIGS. 7A and 7B, which are a schematic cross-sectional view of the heart from above and a schematic side-view in which a portion of the heart and native aortic valve 16 have been nearly laid open, indicating vertical locations at which pacing pulses may be delivered to the heart, in accordance with respective applications of the present invention.

The methods described hereinbelow with reference to FIGS. 6A-C and 7A-B may be performed using prosthetic aortic valve 20, any of the prosthetic aortic valves described in the patents and patent application publications incorporated herein by reference, or other prosthetic aortic valves comprising electrodes, which may be known in the art. Alternatively, these methods may be performed using pacing electrodes inserted into the heart and/or the patient's body, or placed in contact with an external surface of the patient's body, as appropriate.

In these methods, each of the pacing pulses are typically delivered with:
a voltage of at least 0.5 V, no more than 25 V (e.g., no more than 18 V, such as no more than 12 V, e.g., no more than 5 V), and/or 0.5-25 V (e.g., 0.5-18 V, such as 0.5-12 V, e.g., 0.5-5V),
a current of at least 0.2 milliamps (e.g., at least 0.5 milliamps), no more than 50 milliamps (e.g., no more than 36 milliamps), and/or 0.2-50 milliamps (e.g., 0.5-36 milliamps),
a pulse duration of at least 0.3 milliseconds, no more than 0.7 milliseconds, and/or 0.3-0.7 milliseconds, and/or
a pacing frequency of at least 0.5 Hz, no more than 2.5 Hz, and/or 0.5-2.5 Hz.

Typically, the pacing pulses are ventricular pacing pulses. For some applications, the pacing pulses are delivered in VVI mode (Ventricular sensing, Ventricular pacing, Inhibition of pacing when native activity is sensed), DDD mode, VDD mode, or DDDR mode, either with sensing from an implanted electrode or from an external electrode. Alternatively, the pacing pulses may be delivery in another pacing mode, such as any known pacing mode. For other applications, the pacing pulses are delivered in VOO mode (ventricular asynchronous pacing). Optionally, in either the VVI or the VOO mode, rapid pacing is applied.

### Angular Locations

Reference is made to FIGS. 6A-B. In some applications of the present invention, pacing pulses are delivered to one or more of the following angular locations, with respect to an axis 306 of LVOT 302, set forth in Table 1 and schematically labeled in FIG. 6A and/or FIG. 6B, using techniques described hereinbelow. It is noted that the angular anatomical landmarks may or may not be at the same height as the vertical locations described hereinbelow with reference to FIG. 7B; instead, the angular anatomical landmarks are provided to indicate the angular locations around axis 306 of LVOT 302.

TABLE 1

| Angular | |
|---|---|
| Location | Description of Angular Anatomical Landmarks |
| 1 | Adjacent to a membranous septum (MS), below the right lateral end of a |
| | non-coronary cusp (NCC) |
| 2 | Adjacent to the aorto-mitral curtain, below an NCC-left coronary cusp |
| | (LCC) interleaflet triangle |
| 3 | Adjacent to the anterior end of the left fibrous trigone (LFT) and the left |
| | lateral end of the muscular part of the ventricular septum |
| 4 | Adjacent to the muscular part of the ventricular septum, anterior to the |
| | membranous septum (MS), across from the right ventricular outflow tract |
| | (RVOT), below the middle of a right coronary cusp (RCC) |
| 5 | Adjacent to the membranous septum (MS), below the right lateral end of |
| | the RCC |
| 6 | Adjacent to mid-portion of the NCC |
| 7 | Adjacent to the aorto-mitral curtain, below the left-lateral end of the |
| | NCC |
| 8 | Adjacent to the left lateral end of the muscular part of the ventricular |
| | septum closest to the LFT |
| 9 | Adjacent to the muscular part of the ventricular septum, below the mid- |
| | portion of the RCC |
| 10 | Adjacent to the aorto-mitral curtain and the posterior end of the LFT |
| 11 | Adjacent to the mid-portion of the NCC, closer to the NCC-LCC |
| | commissure than to the NCC-RCC commissure |
| 12 | Adjacent to the muscular part of the ventricular septum, below the left |
| | side of the RCC |
| 13 | Adjacent to the muscular part of the ventricular septum, below the right |
| | side of the LCC |

Reference is made to FIG. 6A. In some applications of the present invention, pacing pulses are delivered to one or more of the following angular segments, with respect to axis 306 of LVOT 302, set forth in Table 2 and schematically labeled in FIG. 6A, using techniques described hereinbelow. As also set forth in Table 2, each of the angular segments includes, but is not limited to, one or more of the angular locations set forth in Table 1. It is noted that the angular anatomical landmarks may or may not be at the same height as the vertical locations described hereinbelow with reference to FIG. 7B; instead, the angular anatomical landmarks are provided to indicate the angular locations around axis 306 of LVOT 302.

TABLE 2

| | | Angular |
|---|---|---|
| | | Segment |
| | | includes |
| Angular | | Angular |
| Segment | Description of Angular Anatomical Landmarks | Locations |
| A | Adjacent to the membranous septum (MS) | 1, 5 |
| B | Adjacent to the aorto-mitral curtain, below the | 2, 7 |
| | NCC-LCC interleaflet triangle | |
| C | Adjacent to the LFT | 3, 10 |
| D | Adjacent to the anterior-right side of the muscular | 4, 9, 12 |
| | part of the ventricular septum, below the RCC | |
| E | Adjacent to the muscular part of the ventricular | 8, 13 |
| | septum, below the anterior side of the LCC | |
| F | Adjacent to the RFT (a/k/a as the Central Fibrous | 6, 11 |
| | Body) | |

### Vertical Locations

Reference is made to FIG. 7B. In some applications of the present invention, pacing pulses are delivered at a vertical location between (a) an upper plane 130 corresponding to an aortic annulus plane 310 and (b) a lower plane 132 parallel to aortic annulus plane 310 and at a distance D below aortic annulus plane 310, distance D equal to 8% of a perimeter of an aortic annulus 111. Alternatively, distance D may be equal 10 to 6% or to 4%. For some applications, the vertical location is between (a) a second distance below upper plane 130, the second distance equal to 2.500 of the perimeter of aortic annulus 111, and (b) lower plane 132.

### Horizontal Pacing

Reference is made to FIGS. 6A-B and 7A-B. In an application of the present invention, a method is provided for delivering ventricular pacing pulses to the heart, the method generally comprising placing first and second electrodes in contact with left ventricular endocardium 300 of a left ventricular outflow tract (LVOT) 302 at a first-electrode site and a second-electrode site, respectively. The ventricular pacing pulses are delivered by driving a pacing signal between the first and the second electrodes, optionally by activating circuitry 40 of valve prosthesis system 10, described hereinabove with reference to FIGS. 1A-3I. This pacing protocol is considered “horizontal pacing” because the anode and the cathode are placed at the same distance D below aortic annulus plane 310, or at approximately the same distance D below the aortic annulus plane.

Optionally, in any of the pacing techniques described herein, the pacing signal may be driven between:
(a) a plurality of first electrodes and (b) the second electrode,
(a) the first electrode and (b) a plurality of second electrodes, or
(a) a plurality of first electrodes and (b) a plurality of second electrodes.

For some applications, the method comprises positioning the first electrode in contact with (i.e., in physical contact with) left ventricular endocardium 300 of left ventricular outflow tract (LVOT) 302 at a first-electrode site having:
a first-electrode angular location with respect to axis 306 of LVOT 302, and
a first-electrode vertical location between (a) an upper plane 130 corresponding to an aortic annulus plane 310 and (b) a lower plane 132 parallel to aortic annulus plane 310 and at a distance D below aortic annulus plane 310, distance D equal to 8% of a perimeter of an aortic annulus 111; alternatively, distance D may be equal to 6% or to 4%; for some applications, the vertical location is between (a) a second distance below upper plane 130, the second distance equal to 2.5% of the perimeter of aortic annulus 111, and (b) lower plane 132.

For some applications, the method further comprises positioning the second electrode in contact with (i.e., in physical contact with) left ventricular endocardium 300 of left ventricular outflow tract (LVOT) 302 at a second-electrode site having:
a second-electrode angular location with respect to axis 306 of LVOT 302, the second-electrode angular location different from the first-electrode angular location, and
a second-electrode vertical location between upper plane 130 and lower plane 132, the second-electrode vertical location the same as or different from the first-electrode vertical location; alternatively, distance D may be equal to 6% or to 4%; for some applications, the vertical location is between (a) a second distance below upper plane 130, the second distance equal to 2.5% of the perimeter of aortic annulus 111, and (b) lower plane 132.

As used in the present application, including in the claims:
aortic annulus plane 310 is the plane defined by respective nadirs 112A, 112B, and 112C of a non-coronary cusp (NCC) 114A, a right coronary cusp (RCC) 114B, and a left coronary cusp (LCC) 114C (although actually planar, upper and lower planes 130 and 132 are indicated by curved lines in FIG. 6B because the intersections between these planes and left ventricular endocardium 300 of LVOT 302 define respective curves in the nearly laid open view of FIG. 6B);
aortic annulus 111 is the quasi-circular tissue of LVOT 302 at aortic annulus plane 310;
an “angular location with respect to axis 306 of LVOT 302” means an angular location around axis 306, i.e., an “o'clock” of the location around the axis, as shown, for example, in FIG. 6A; and
“vertical” means along a vertical axis that passes vertically from inferior to superior, as defined in human anatomy.

As is known in cardiac anatomy, MS 108 includes both an atrioventricular portion and an inter-ventricular portion.

Alternatively, the distance D is 10 mm, 6 mm, or 4 mm.

Reference is still made to FIGS. 6A-B and 7A-B. For some applications, the ventricular pacing pulses are delivered between one of the following pairs of first-electrode angular locations and second-electrode angular locations set forth in Tables 3 and 4, respectively at the first-electrode vertical location and the second-electrode vertical location described above. Either the first electrode is configured as a cathode and the second electrode as an anode, or the first electrode is configured as an anode and the second electrode as a cathode. Each of the pairs of angular locations in Table 3 includes at least one angular segment, and each of the pairs of angular locations in Table 4 includes two angular locations.

TABLE 3

| First-Electrode Angular Location | Second-Electrode Angular Location |
|---|---|
| along Angular Segment A | along Angular Segment B |
| along Angular Segment A | along Angular Segment C |
| along Angular Segment A | at Angular Location 6 |
| along Angular Segment A | at Angular Location 8 |
| along Angular Segment B | along Angular Segment D |
| along Angular Segment B | at Angular Location 6 |
| along Angular Segment B | at Angular Location 8 |
| along Angular Segment D | at Angular Location 8 |
| along Angular Segment E | along Angular Segment D |

TABLE 4

| First-Electrode Angular Location | Second-Electrode Angular Location |
|---|---|
| at Angular Location 1 | at Angular Location 3 |
| at Angular Location 1 | at Angular Location 5 |
| at Angular Location 1 | at Angular Location 10 |
| at Angular Location 3 | at Angular Location 5 |
| at Angular Location 5 | at Angular Location 6 |
| at Angular Location 5 | at Angular Location 7 |
| at Angular Location 5 | at Angular Location 8 |
| at Angular Location 5 | at Angular Location 10 |
| at Angular Location 6 | at Angular Location 7 |
| at Angular Location 6 | at Angular Location 8 |
| at Angular Location 7 | at Angular Location 8 |
| at Angular Location 7 | at Angular Location 9 |
| at Angular Location 8 | at Angular Location 9 |
| at Angular Location 12 | at Angular Location 13 |
| at Angular Location 13 | at Angular Location 9 |

Reference is made to FIGS. 1A-3I, 6A-B, and 7A-B. For some applications in which the method is performed using prosthetic aortic valve 20, the first electrode is one of electrodes 34 of prosthetic aortic valve 20, such as first electrode 34A, which is disposed at a distal upstream portion of prosthetic aortic valve 20. The desired first-electrode angular location may be achieved by rotating prosthetic aortic valve 20 during deployment of the valve, and the desired first-electrode vertical location may be achieved by adjusting the vertical (axial) position of prosthetic aortic valve 20 during deployment of the valve.

Reference is still to FIGS. 1A-3I, 6A-B, and 7A-B. For some applications, positioning the first electrode comprises:
delivering a frame, such as frame 30, described hereinabove with reference to FIGS. 1A-3I, to native aortic valve 16 in a constrained delivery configuration, the frame including interconnected stent struts 190; the first electrode is coupled to the frame; and
transitioning the frame to an expanded deployment configuration, in which the frame positions and holds the first electrode in contact with left ventricular endocardium 300 of LVOT 302.

For example, the first electrode may be one of electrodes 34 of prosthetic aortic valve 20, such as first electrode 34A, which is disposed at a distal upstream portion of prosthetic aortic valve 20.

For some of these applications, the second electrode is coupled to the frame. The second electrode is positioned in electrical communication with the patient's body by transitioning the frame to the expanded deployment configuration, in which the frame positions and holds the second electrode in contact with left ventricular endocardium 300 of LVOT 302 at the second-electrode angular location and the second-electrode vertical location.

For example, the second electrode may be one of electrodes 34 of prosthetic aortic valve 20, such as another first electrode 34A disposed at a distal upstream portion of prosthetic aortic valve 20 (in which case, electrodes 34 include at least two first electrodes 34A).

For some applications, for delivering ventricular pacing pulses, the first-electrode angular location is selected from the group of angular locations consisting of: (a) an angular location, with respect to axis 306 of LVOT 302, adjacent to a right side of the muscular part of a ventricular septum, below the RCC (Angular Segment D), and (b) an angular location, with respect to axis 306 of LVOT 302, adjacent to the muscular part of a ventricular septum, below an anterior side of the LCC (Angular Segment E). In other words, the first-electrode angular extends, with respect to axis 306 of LVOT 302, inclusively, from (a) adjacent to the muscular part of the ventricular septum, anterior to a membranous septum (MS), across from the right ventricular outflow tract (Angular Location 4), (b) around a portion of the RCC, (c) around a portion of the LCC, to (d) adjacent to the left lateral end of the muscular part of a ventricular septum closest to the left fibrous trigone (Angular Location 8).

For some of these application, the first electrode is configured as a cathode and the second electrode as an anode.

For some of these applications, the second-electrode angular location is selected from the group of angular locations consisting of: (a) an angular location, with respect to axis 306 of LVOT 302, adjacent to a right side of the muscular part of a ventricular septum, below the RCC (Angular Segment D), and (b) an angular location, with respect to axis 306 of LVOT 302, adjacent to the muscular part of a ventricular septum, below an anterior side of the LCC (Angular Segment E).

Experimental evidence demonstrating successful horizontal ventricular pacing is provided hereinbelow in the descriptions of Experiments #2, #3, and #5.

In some applications of the present invention, a method is provided for delivering atrial pacing pulses to a heart of a patient. The method may optionally implement any of the techniques described herein, including, but not limited to, the techniques described hereinabove in the present section entitled, “Horizontal Pacing.” For some applications, the method comprises positioning the first electrode in contact with (i.e., in physical contact with) left ventricular endocardium 300 of left ventricular outflow tract (LVOT) 302 at a first-electrode site having:
a first-electrode angular location with respect to axis 306 of LVOT 302, and
a first-electrode vertical location between (a) upper plane 130 corresponding to aortic annulus plane 310 and (b) lower plane 132 parallel to aortic annulus plane 310 and at distance D below aortic annulus plane 310, distance D equal to 8% of a perimeter of an aortic annulus 111; alternatively, distance D may be equal to 6% or to 4%; for some applications, the vertical location is between (a) a second distance below upper plane 130, the second distance equal to 2.5% of the perimeter of aortic annulus 111, and (b) lower plane 132.

For some applications, the method further comprises positioning the second electrode in contact with (i.e., in physical contact with) left ventricular endocardium 300 of left ventricular outflow tract (LVOT) 302 at a second-electrode site having:
a second-electrode angular location with respect to axis 306 of LVOT 302, the second-electrode angular location different from the first-electrode angular location, and
a second-electrode vertical location between upper plane 130 and lower plane 132, the second-electrode vertical location the same as or different from the first-electrode vertical location; alternatively, distance D may be equal to 6% or to 4%; for some applications, the vertical location is between (a) a second distance below upper plane 130, the second distance equal to 2.5% of the perimeter of aortic annulus 111, and (b) lower plane 132.

For some applications, for delivering atrial pacing pulses, the first-electrode angular location is along a partially left angular segment 122 (labeled in FIG. 6D) extending, with respect to axis 306 of LVOT 302, inclusively, from (a) adjacent to the anterior end of the LFT and the left lateral end of the muscular part of the ventricular septum (Angular Location 3), (b) around a left posterior portion of LVOT 302, to (c) below a middle of the NCC.

Experimental evidence demonstrating successful horizontal atrial pacing is provided hereinbelow in the descriptions of Experiments #5 and #7.

### Vertical Pacing

Reference is made to FIGS. 6C and 7A-B. In an application of the present invention, a method is provided for delivering ventricular pacing pulses to the heart, the method generally comprising placing a first electrode in contact with left ventricular endocardium 300 of left ventricular outflow tract (LVOT) 302 at a first-electrode site, and a second electrode at a second-electrode site in electrical communication with the patient's body, such as in the aorta (optionally angularly aligned with first-electrode angular location), elsewhere within the patient's body (such as in contact with the heart, e.g., pericardium of the heart), or on an external surface of skin of the patient's body, e.g., using a patch electrode). The ventricular pacing pulses are delivered by driving a pacing signal between the first and the second electrodes, optionally by activating circuitry 40 of valve prosthesis system 10, described hereinabove with reference to FIGS. 1A-3I. This pacing protocol is considered “vertical pacing” because one of the electrodes is placed above aortic annulus plane 310 and the other electrode is placed below aortic annulus plane 310.

For some applications, the method comprises positioning the first electrode in contact with (i.e., in physical contact with) left ventricular endocardium 300 of left ventricular outflow tract (LVOT) 302 at a first-electrode site having:
a first-electrode angular location with respect to axis 306 of LVOT 302, and
a first-electrode vertical location between (a) upper plane 130 corresponding to aortic annulus plane 310 and (b) lower plane 132 parallel to aortic annulus plane 310 and at distance D below aortic annulus plane 310, distance D equal to 8% of a perimeter of an aortic annulus 111; alternatively, distance D may be equal to 6% or to 4%; for some applications, the vertical location is between (a) a second distance below upper plane 130, the second distance equal to 2.5% of the perimeter of aortic annulus 111, and (b) lower plane 132.

Reference is still made to FIGS. 6C and 7A-B. For some applications, the ventricular pacing pulses are delivered between (a) one of the following first-electrode angular locations set forth in Tables 5 and 6 and (b) the second-electrode site in the aorta, optionally angularly aligned with first-electrode angular location. Either the first electrode is configured as a cathode and the second electrode as an anode, or the first electrode is configured as an anode and the second electrode as a cathode. Each of the locations in Table 5 includes one angular segment, and each of the angular locations in Table 6 includes one angular location.

TABLE 5
First-Electrode Angular Location

along Angular Segment A
along Angular Segment B
along Angular Segment D

TABLE 6
First-Electrode Angular Location

at Angular Location 1
at Angular Location 2
at Angular Location 4
at Angular Location 6
at Angular Location 8

Reference is made to FIGS. 1A-3I, 6C, and 7A-B. For some applications in which the method is performed using prosthetic aortic valve 20, the first electrode is one of electrodes 34 of prosthetic aortic valve 20, such as first electrode 34A, which is disposed at a distal upstream portion of prosthetic aortic valve 20. The desired first-electrode angular location may be achieved by rotating prosthetic aortic valve 20 during deployment of the valve, and the desired first-electrode vertical location may be achieved by adjusting the vertical (axial) position of prosthetic aortic valve 20 during deployment of the valve.

### Interleaflet Triangles

Reference is made to FIG. 7B. In some applications of the present invention, the first-electrode site is on an interleaflet triangle 134 of native aortic valve 16. Optionally, the first-electrode site is closer to aortic annulus plane 310 than to a plane defined by respective commissures of the three cusps of native aortic valve 16 (i.e., the first-electrode site is in a lower half of one of the interleaflet triangles 134).

The second-electrode site is in electrical communication with within a body of the patient, such as in the aorta (optionally angularly aligned with first-electrode angular location), elsewhere within the patient's body (such as in contact with the heart, e.g., pericardium of the heart), or on an external surface of skin of the patient's body, e.g., using a patch electrode). The ventricular pacing pulses a delivered by driving a pacing signal between the first and the second electrodes.

For some applications, the second-electrode site is in the aorta, for performing “vertical pacing,” such as described hereinabove, mutatis mutandis. Optionally, the second electrode is positioned at the second-electrode site in the aorta angularly aligned with first-electrode site.

For some applications, the interleaflet triangle is a first interleaflet triangle, and the second-electrode site is on a second interleaflet triangle of the aortic valve.

For other applications, the second-electrode site is at a (a) at one of the Angular Locations set forth in Table 1 hereinabove and/or along one of the Angular Segments set forth in Table 2 hereinabove, and (b) a vertical location between upper plane 130 and lower plane 132, as described hereinabove with reference to FIG. 7B.

Experimental evidence demonstrating successful interleaflet triangle pacing is provided hereinbelow in the description of Experiment #4.

### Horizontal Sensing

Reference is now made to FIG. 6D, which is a schematic cross-sectional illustration of native aortic valve 16 from above, indicating angular locations and segments at which electrical activity of the heart may be sensed, in accordance with respective applications of the present invention. Reference is also again made to FIGS. 7A-B.

In some applications of the present invention, a method is provided for sensing electrical activity of the heart, the method generally comprising placing first and second electrodes in contact with left ventricular endocardium 300 of a left ventricular outflow tract (LVOT) 302 at a first-electrode site and a second-electrode site, respectively. The electrical activity of the heart is sensed between the first and the second electrodes (i.e., bipolar sensing), optionally by activating circuitry 40 of valve prosthesis system 10, described hereinabove with reference to FIGS. 1A-3I. Typically, circuitry 40 is activated to attempt to identify a ventricular activation signal and/or an atrial activation signal in an intracardiac electrogram (EGM) sensed using the first and the second electrodes. This sensing protocol is considered “horizontal sensing” because the anode and the cathode are placed at the same distance D below aortic annulus plane 310, or at approximately the same distance D below the aortic annulus plane.

For some applications, circuitry 40 is configured to attempt to identify the intrinsic ventricular activation signal and/or the atrial activation signal in the intracardiac EGM by sensing using only the first and the second electrodes.

For some applications, circuitry 40 is configured to identify the intrinsic ventricular activation signal and/or the atrial activation signal in the intracardiac EGM by configuring the first and the second electrodes as a cathode and as an anode, respectively. Alternatively, for some applications, circuitry 40 is configured to identify the intrinsic ventricular activation signal and/or the atrial activation signal in the intracardiac EGM by configuring the first and the second electrodes as an anode and as a cathode, respectively.

These sensing techniques may implement any of the techniques described herein for pacing, mutatis mutandis, including techniques for positioning the electrodes.

Experimental evidence demonstrating successful horizontal sensing is provided hereinbelow in the descriptions of Experiments #6 and #7. In general, the experimental evidence demonstrates that the polarity of the two electrodes (which is the cathode and which is the anode) does not have a substantial effect on the signal quality (reversing the polarity of the electrodes simply reverses the signal polarity).

Optionally, in any of the sensing techniques described herein, the electrical activity may be sensed between:
(a) a plurality of first electrodes and (b) the second electrode,
(a) the first electrode and (b) a plurality of second electrodes, or
(a) a plurality of first electrodes and (b) a plurality of second electrodes.

For some applications, the method comprises positioning the first electrode in contact with (i.e., in physical contact with) left ventricular endocardium 300 of left ventricular outflow tract (LVOT) 302 at a first-electrode site having:
a first-electrode angular location with respect to axis 306 of LVOT 302, and
a first-electrode vertical location between (a) an upper plane 130 corresponding to an aortic annulus plane 310 and (b) a lower plane 132 parallel to aortic annulus plane 310 and at a distance D below aortic annulus plane 310, distance D equal to 8% of a perimeter of an aortic annulus 111; alternatively, distance D may be equal to 6% or to 4%; for some applications, the vertical location is between (a) a second distance below upper plane 130, the second distance equal to 2.5% of the perimeter of aortic annulus 111, and (b) lower plane 132.

For some applications, the method further comprises positioning the first electrode in contact with (i.e., in physical contact with) left ventricular endocardium 300 of left ventricular outflow tract (LVOT) 302 at a second-electrode site having:
a second-electrode angular location with respect to axis 306 of LVOT 302, the second-electrode angular location different from the first-electrode angular location, and
a second-electrode vertical location between upper plane 130 and lower plane 132, the second-electrode vertical location the same as or different from the first-electrode vertical location; alternatively, distance D may be equal to 6% or to 4%; for some applications, the vertical location is between (a) a second distance below upper plane 130, the second distance equal to 2.5% of the perimeter of aortic annulus 111, and (b) lower plane 132.

In some applications of the present invention:
the first-electrode site has (i) the first-electrode vertical location described immediately above, and (ii) a first-electrode angular location, with respect to axis 306 of LVOT 302, along a partially anterior angular segment 120 extending, with respect to axis 306 of LVOT 302, inclusively, from (a) the posterior end of a membranous septum (MS), (b) around an anterior portion of LVOT 302, to (c) the anterior end of the LFT, and
the second-electrode site has (i) the second-electrode vertical location described immediately above, and (ii) a second-electrode angular location, with respect to axis 306 of LVOT 302, along partially anterior angular segment 120, the second-electrode angular location different from the first-electrode angular location.

For some of these applications, circuitry 40 is activated to sense an intrinsic (i.e., non-pacing-induced) ventricular activation signal and/or atrial activation signal in the intracardiac EGM. For others of these applications, circuitry 40 is activated to sense a pacing-induced ventricular activation signal and/or atrial activation signal in the intracardiac EGM.

For some applications, the first-electrode angular location is along a sub-segment of the partially anterior angular segment 120, the sub-segment extending, with respect to axis 306 of LVOT 302, inclusively, from (a) adjacent to the membranous septum (MS), below a right lateral end of the NCC (Angular Location 1), (b) around the anterior portion of LVOT 302, to (c) the anterior end of the LFT.

For some applications, the first-electrode angular location is along a sub-segment of partially anterior angular segment 120, the sub-segment extending, with respect to axis 306 of LVOT 302, inclusively, from (a) the posterior end of membranous septum (MS), (b) around the anterior portion of LVOT 302, to (c) adjacent to the left lateral end of the muscular part of the ventricular septum closest to a left fibrous trigone (LFT) (Angular Location 8). For some of these applications, the first-electrode angular location is along a sub-segment of the partially anterior angular segment, the sub-segment extending, with respect to axis 306 of LVOT 302, inclusively, from (a) adjacent to the membranous septum (MS), below a right lateral end of the NCC, (b) around the anterior portion of the LVOT, to (c) adjacent to the left lateral end of the muscular part of the ventricular septum closest to a left fibrous trigone (LFT) (Angular Location 8).

For some applications, the second-electrode angular location is offset from the first-electrode angular location by at least 10 degrees, such as at least 20 degrees, with respect to axis 306 of LVOT 302.

For some applications, the first-electrode angular location is adjacent to the left lateral end of the muscular part of a ventricular septum closest to a left fibrous trigone (Angular Location 8). For some of these applications, the second-electrode angular location is:
adjacent to the muscular part of a ventricular septum, below a left side of the RCC (Angular Location 12),
adjacent to the muscular part of a ventricular septum, below the mid-portion of the RCC (Angular Location 9), or
adjacent to a membranous septum (MS), below a right lateral end of the NCC (Angular Location 5).

For some applications, the first-electrode angular location is adjacent to the muscular part of a ventricular septum, below a left side of the RCC (Angular Location 12). For some of these applications, the second-electrode angular location is:
adjacent to a membranous septum (MS), below a right lateral end of the NCC (Angular Location 5), or
adjacent to a membranous septum (MS), below a right lateral end of the RCC (Angular Location 1).

For some applications, the first-electrode angular location is adjacent to the muscular part of a ventricular septum, below the mid-portion of the RCC (Angular Location 9).

For some applications, the first-electrode angular location is adjacent to a membranous septum (MS), below a right lateral end of the NCC (Angular Location 5).

For some applications, the first-electrode angular location is adjacent to a membranous septum (MS), below a right lateral end of the RCC (Angular Location 1).

In some applications of the present invention:
the first-electrode site has (i) the first-electrode vertical location described above, and (ii) a first-electrode angular location, with respect to axis 306 of LVOT 302, along partially left angular segment 122 extending, with respect to axis 306 of LVOT 302, inclusively, from (a) adjacent to the anterior end of the left fibrous trigone (LFT) and the left lateral end of the muscular part of a ventricular septum (Angular Location 3), (b) around a left posterior portion of LVOT 302, to (c) below a middle of the NCC; and
the second-electrode site has (i) the second-electrode vertical location described above, and (ii) a second-electrode angular location, with respect to axis 306 of LVOT 302, along partially left angular segment 122, the second-electrode angular location different from the first-electrode angular location.

For some applications, the second-electrode angular location is offset from the first-electrode angular location by at least 10 degrees, such as at least 20 degrees, e.g., at least 30 degrees, with respect to axis 306 of LVOT 302.

For some applications, the first-electrode angular location is adjacent to an anterior end of the left fibrous trigone and a left lateral end of the muscular part of a ventricular septum (Angular Location 3). For some of these applications, the second-electrode angular location is adjacent to an aorto-mitral curtain and a posterior end of the left fibrous trigone (Angular Location 7).

For some applications, the first-electrode angular location is adjacent to an aorto-mitral curtain and a posterior end of the left fibrous trigone (Angular Location 7). For some of these applications, the second-electrode angular location is below the middle of the NCC (Angular Location 11).

For some applications, the first-electrode angular location is below the middle of the NCC (Angular Location 11).

In some applications of the present invention:
the first-electrode site has (i) the first-electrode vertical location described above, and (ii) a first-electrode angular location, with respect to axis 306 of LVOT 302, along a partially anterior angular segment 124 extending, with respect to axis 306 of LVOT 302, inclusively, from (a) adjacent to a membranous septum (MS), below the right lateral end of the RCC, (b) around an anterior portion of LVOT 302, to (c) adjacent to an RCC-LCC commissure 126; and
the second-electrode site has (i) the second-electrode vertical location described above, and (ii) a second-electrode angular location, with respect to axis 306 of LVOT 302, along a partially left angular segment extending, with respect to axis 306 of LVOT 302, inclusively, from (a) adjacent to an anterior end of a left fibrous trigone (LFT) and a left lateral end of the muscular part of a ventricular septum, (b) around a left posterior portion of LVOT 302, to (c) below a middle of the NCC.

For some applications, such as for sensing a ventricular activation signal, the first-electrode angular location is (a) adjacent to a right side of the muscular part of a ventricular septum, below a right coronary cusp (RCC) (Angular Segment D), (b) adjacent to the muscular part of a ventricular septum, below an anterior side of a left coronary cusp (LCC) (Angular Segment E), or (c) adjacent to a membranous septum (Angular Segment A).

For some applications, such as for sensing an atrial activation signal, the first-electrode angular location is along a segment that extends, with respect to an axis of the LVOT, inclusively, from (a) adjacent to an anterior end of a left fibrous trigone (LFT) and a left lateral end of the muscular part of a ventricular septum (Angular Location 3), (b) around a posterior portion of the LVOT, to (c) adjacent to a membranous septum (MS), below a right lateral end of a right coronary cusp (RCC) (Angular Location 5).

### Generally-Applicable Techniques

Reference is made to FIGS. 1A-3I, 6A-D, and 7A-B. For some applications, positioning the first electrode comprises:
delivering a frame, such as frame 30, described hereinabove with reference to FIGS. 1A-3I, to native aortic valve 16 in a constrained delivery configuration, the frame including interconnected stent struts 190; the first electrode is coupled to the frame; and
transitioning the frame to an expanded deployment configuration, in which the frame positions and holds the first electrode in contact with left ventricular endocardium 300 of LVOT 302.

For example, the first electrode may be one of electrodes 34 of prosthetic aortic valve 20, such as first electrode 34A, which is disposed at a distal upstream portion of prosthetic aortic valve 20.

For some of these applications, the second electrode is coupled to the frame. The second electrode is positioned in electrical communication with the patient's body by transitioning the frame to the expanded deployment configuration, in which the frame positions and holds the second electrode at a second-electrode vertical location above (i.e., superior to) aortic annulus plane 310 in contact with blood and not in contact with an aortic wall. For example, the second electrode may be one of electrodes 34 of prosthetic aortic valve 20, such as second electrode 34B, which is disposed at a proximal downstream portion of prosthetic aortic valve 20.

For some applications, positioning first electrode 34A at the first-electrode site and positioning second electrode 34B at the second-electrode site comprise:
delivering a support to an aortic position in the heart in a constrained delivery configuration; first and second electrodes 34A and 34B are coupled to the support; and
transitioning the support to an expanded deployment configuration, in which the support positions and holds first and second electrodes 34A and 34B in contact with left ventricular endocardium 300 of the LVOT 302 at the first-electrode and the second-electrode sites, respectively.

For some of these applications, the method further comprising introducing a prosthetic aortic valve into a body of the patient and placing the prosthetic aortic valve within the support, the prosthetic aortic valve including a plurality of prosthetic leaflets arranged so as to allow blood flow in a downstream direction and inhibit blood flow in an upstream direction.

For other applications, positioning the second electrode in electrical communication with the patient's body comprises positioning the second electrode elsewhere in the patient's body (such as in contact with the heart, e.g., with pericardium of the heart), or on an external surface of skin of the patient's body, e.g., using a patch electrode (configuration not shown).

Reference is still made to FIGS. 1A-3I, 6A-D, and 7A-B. For some applications, delivering the ventricular pacing pulses comprises configuring the first electrode as an anode and the second electrode a cathode. For other applications, delivering the ventricular pacing pulses comprises configuring the first electrode as a cathode and the second electrode an anode.

Experimental evidence demonstrating successful vertical pacing is provided hereinbelow in the descriptions of Experiments #1 and #2.

### EXPERIMENTAL EVIDENCE

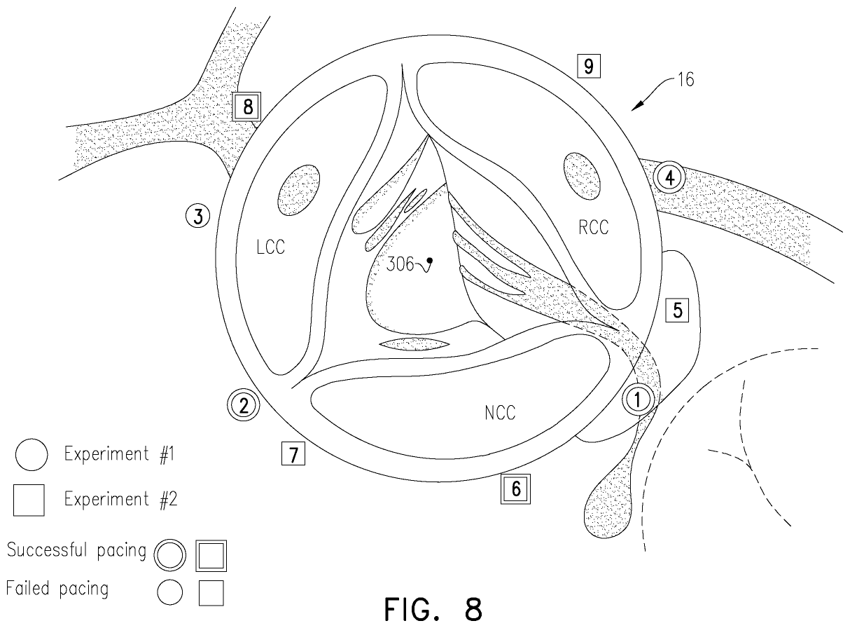

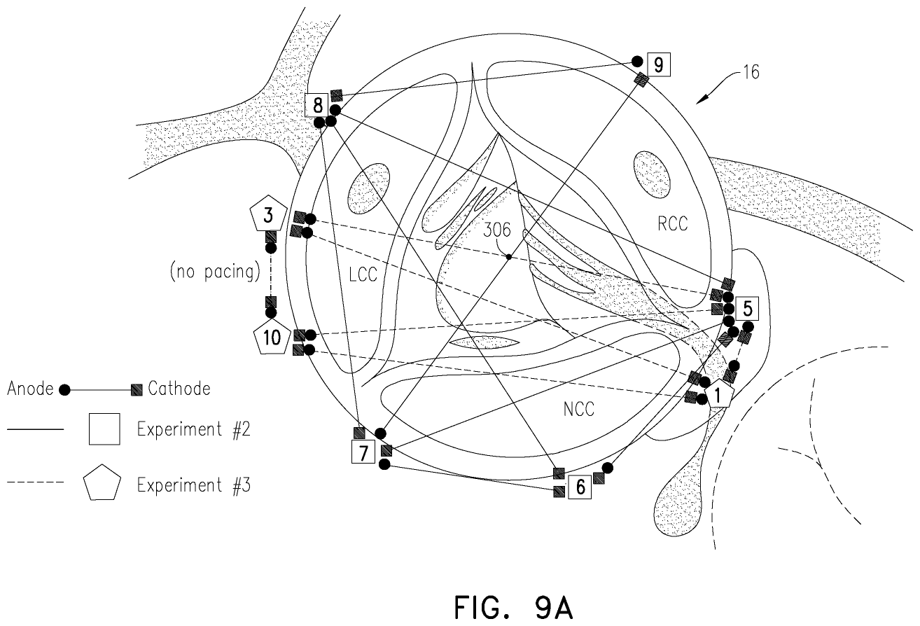

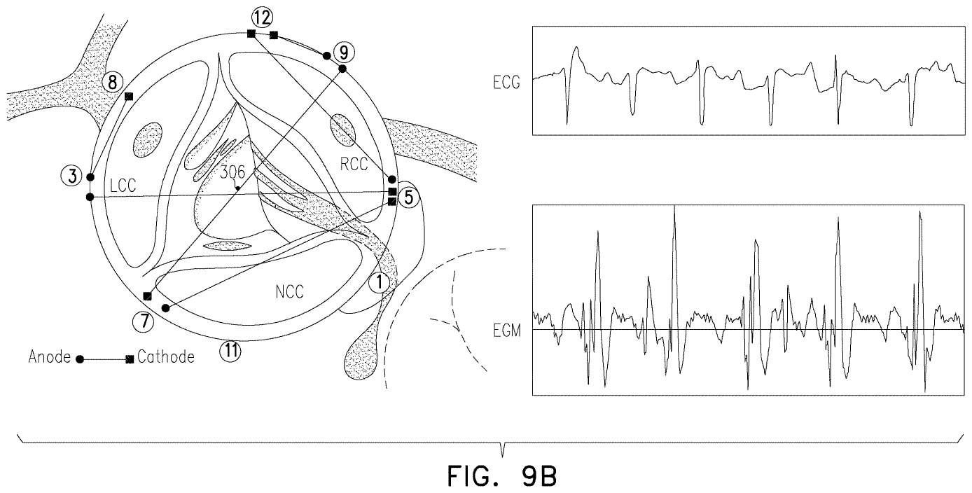

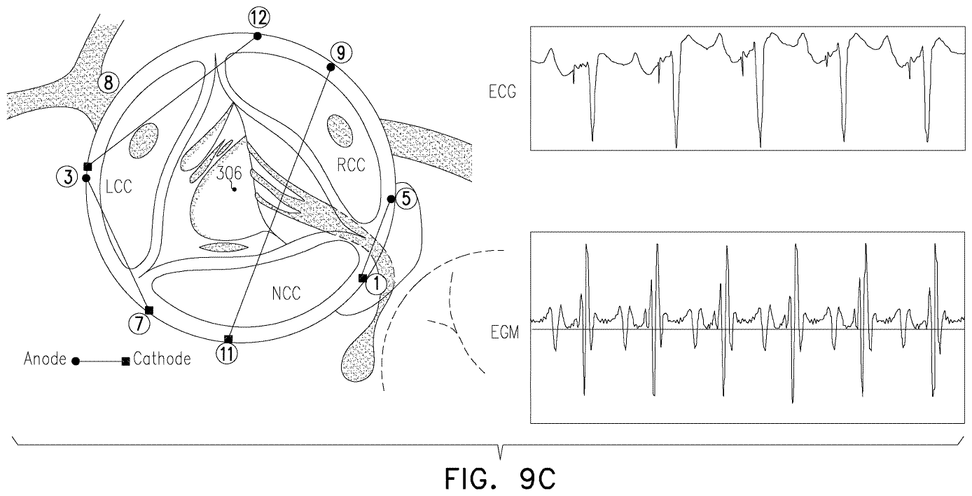

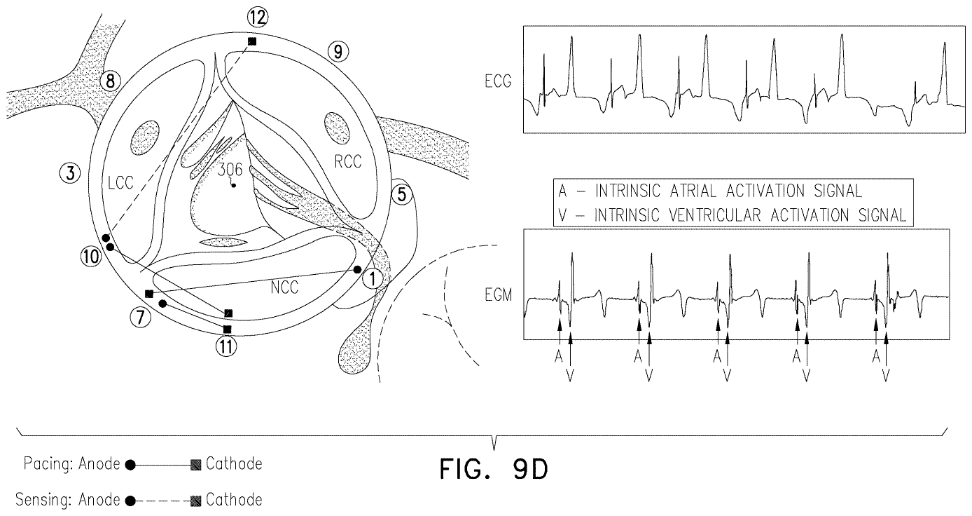

Reference is now made to FIGS. 8 and 9A-D, which are schematic illustrations showing results of five experiments conducted by the inventors, using techniques similar to some of those described herein. Any of the techniques described in these experiments may optionally be incorporated into the techniques of the inventions described herein, as appropriate.

### Experiment #1

A first experiment conducted by the inventors was designed to assess the minimal pacing voltage for ventricular cardiac pacing using electrodes disposed at various electrode locations on an external surface of a frame of a prosthetic aortic valve similar to prosthetic aortic valve 20 described hereinabove with reference to FIGS. 1A-3I. This experiment was designed to assess “vertical pacing,” as described hereinabove with reference to FIGS. 1A-3I, 6C, and 7A-B. The prosthetic aortic valve had a diameter of 27 mm, and was implanted transapically in the native aortic valves of two pigs having weights of 92 kg and 94 kg. (A pig model is widely recognized for its close similarity to humans.)

The results of Experiment #1 are summarized schematically in FIG. 8, in which the pacing locations of this experiment are schematically indicated by circled numbers 1, 2, 3, and 4, using the numbers for the angular locations set forth hereinabove in Table 1.

Pacing was attempted, each time through a different pair of electrodes, and the minimal voltage for ventricular pacing (as registered through an external ECG device) was recorded. Two different pulse generators were used: Osypka 101 PG (Osypka Medical, Berlin, Germany) (output given in volts); and the Medtronic 5392 PG (Medtronic, Minneapolis, Minnesota) (output given in mA). The electrodes were directly, non-wirelessly connected to the pulse generators. Power was calculated as the product of the outputted volts and outputted mA. The pulse generators were configured to pace higher than the intrinsic cardiac rate of the animals, in an asynchronous mode. Pairs of matching leads were connected to the two pulse generators. For each pair of leads, the minimal pacing voltage at which the set pace dominated the animal's cardiac rate (as registered in the ECG) was measured.

Each pair of electrodes included an anode and a cathode. One of two electrodes was disposed super-annularly on the frame of the prosthetic aortic valve, angularly aligned with the other of the two electrodes. The other of the two electrodes was disposed sub-annularly, approximately 4-8 mm below the annulus, at Angular Locations 1, 2, 3, and 4 set forth in Table 1 hereinabove.

The following Table 7 summarizes the results at each of the cathode Angular Locations, using the numbers set forth in Table 1 hereinabove:

TABLE 7

| Cathode Angular | Min. voltage for | Min. current for | Min. power for |
|---|---|---|---|
| Location | pacing [V] | pacing [mA] | pacing [mW] |
| 4 | Unknown (>12) | 7-8 | Unknown, |
| | | | estimated ~112 |
| 1 | 3 | 1.5 | 4.5 |
| 3 | — | — | — |
| 2 | 8 | 3.5 | 28 |

As can be seen in Table 7, successful ventricular pacing was achieved at Angular Locations 1, 2, and 3. The level of contact between the electrode ends and the tissue was not measured. Insufficient contact between the exposed electrode and the tissue may explain the high variability in power threshold between the angular locations; however, no evidence for this was identified.

### Experiment #2

A second experiment conducted by the inventors was designed to assess the minimal pacing voltage for ventricular cardiac pacing using electrodes disposed at various electrode locations on an external surface of a frame of a prosthetic aortic valve similar to prosthetic aortic valve 20 described hereinabove with reference to FIGS. 1A-3I. This experiment was designed to assess both “vertical pacing” and “horizontal pacing,” as described hereinabove with reference to FIGS. 1A-3I, 6A-B, and 7A-B. Other than as described below, the second experiment was conducted in a similar manner to Experiment #1 described hereinabove. The electrodes comprised exposed wire leads, which were directly, non-wirelessly connected to the pulse generators. The prosthetic aortic valve had a diameter of 27 mm, and was implanted transapically in the native aortic valves of two pigs having weights of about 90 kg.

The results of Experiment #2 are summarized schematically partially in FIG. 8 and partially in FIG. 9A, in which the pacing locations of this experiment are schematically indicated by numbers 5, 6, 7, 8, and 9 in squares, using the numbers set forth in Table 1 hereinabove.

In a first set of electrode placements similar to those described in Experiment #1, “vertical pacing” was performed. Each pair of electrodes included an anode and a cathode. The anode was disposed super-annularly on the frame of the prosthetic aortic valve, angularly aligned with the cathode. The cathode was disposed sub-annularly, approximately 3-5 mm below the annulus, at Angular Locations 5, 6, 7, 8, and 9 set forth in Table 1 hereinabove.

The following Table 8 summarizes the results at each of the cathode Angular Locations, using the numbers set forth in Table 1 hereinabove:

TABLE 8

| Cathode | | | |
|---|---|---|---|
| Angular | Min. voltage for | Min. current for | Min. power for |
| Location | pacing [V] | pacing [mA] | pacing [mW] |
| 9 | No pacing (>18 V) | | |
| 6 | 13 | 28.9* | 375.6* |
| 5 | No pacing (>18 V) | | |
| 8 | 6 | 13.3* | 80* |
| 7 | No pacing (>18 V) | | |

*Values estimated based on impedance derived from nearby measurements

As can be seen in Table 8, successful pacing was achieved at Angular Locations 6 and 8.

In a second set of electrode placements, “horizontal pacing” was performed. Each pair of electrodes included an anode and a cathode, both of which were disposed sub-annularly, approximately 3-5 mm below the annulus, at two different angular locations. Each pair of electrodes is schematically labeled in FIG. 9A by a connecting line, the ends of which indicate the angular locations of the anode and the cathode. As indicated in the key in FIG. 9A, the endpoints of the lines indicate which electrode was an anode (by a circle) and which electrode was a cathode (by a square), or that both sets of polarities were tested (by both circles and squares at both endpoints of the lines). (The lines connect the electrode pairs only schematically, and may or may not indicate the actual conduction paths between the electrode pairs, which were not measured in the experiment.)

The following Table 9 summarizes the results for each of the pairs of Angular Locations, using the numbers set forth in Table 1 hereinabove:

TABLE 9

| Anode Angular | Cathode Angular | Min. voltage for |
|---|---|---|
| Location | Location | pacing [V] |
| 9 | 8 | 9.5 |
| 7 | 9 | 17 |
| 6 | 5 | 5.6 |
| 5 | 7 | 7 |
| 8 | 5 | 6 |
| 8 | 6 | 12 |
| 7 | 6 | 12 |
| 8 | 7 | 10 |
| 5 | 6 | 5.6 |

As can be seen, successful pacing was achieved at all pairs of Angular Locations. The best pacing, as indicated by the minimum voltage required to achieve pacing, was achieved at the following pairs of Angular Locations (Anode-Cathode respectively): 5-6, 6-5, 8-5, and 5-7.

Observed values for pacing [V] and impedance [ohms] were overall consistent through repeated measurement. However, a minor decline in performance over consecutive measurements was observed: the second and third pacing attempts, where performed, required slightly stronger stimulation output to achieve pacing.

### Experiment #3

A third experiment conducted by the inventors was designed to assess the minimal pacing voltage for ventricular cardiac pacing using electrodes disposed at various electrode locations on an external surface of a frame of a prosthetic aortic valve similar to prosthetic aortic valve 20 described hereinabove with reference to FIGS. 1A-3I. Other than as described below, the third experiment was conducted in a similar manner to the horizontal pacing portion of Experiment #2 described hereinabove. The prosthetic aortic valve had a diameter of 27 mm, and was implanted transapically in the native aortic valve of one pig having a weight of 104 kg.

The results of Experiment #3 are summarized schematically in FIG. 9A, in which the pacing locations of this experiment are schematically indicated by numbers 1, 3, 5, and 10 in pentagons, using the numbers set forth in Table 1 hereinabove.

Each pair of electrodes included an anode and a cathode, both of which were disposed sub-annularly, approximately 4-6 mm below the annulus, at two different angular locations. Unlike in Experiments #1 and #2, in Experiment #3 an external transmitter was used to wirelessly transmit energy to the implant, which delivered the energy as DC current stimulation to the tissue through the selected electrodes. The system enabled, by wireless commands from the external transmitter, configuration of each electrode to be a cathode or an anode.

Each pair of electrodes is schematically labeled in FIG. 9A by a connecting line, the ends of which indicate the angular locations of the anode and the cathode. As indicated in the key in FIG. 9A, the endpoints of the lines indicate which electrode was an anode (by a circle) and which electrode was a cathode (by a square), or that both sets of polarities were tested (by both circles and squares at both endpoints of the lines). (The lines connect the electrode pairs only schematically, and may or may not indicate the actual conduction paths between the electrode pairs, which were not measured in the experiment.)

The following Table 10 summarizes the results for each of the pairs of Angular Locations, using the numbers set forth in Table 1 hereinabove; as mentioned above, each pair was tested twice, in both directions of stimulation, and is thus listed twice in the table. The pacing currents presented in Table 10 represent the current that flowed through the induction coil of the transmitter in the external transmitter, rather than the current applied to the cardiac tissue by the electrodes; thus, the values cannot be directly compared to the values presented in Tables 7, 8, and 9 for Experiments #1 and #2.

TABLE 10

| Anode Angular | Cathode Angular | Min. current for |
|---|---|---|
| Location | Location | pacing [A] |
| 10 | 5 | 1.8 |
| 5 | 1 | 4.2 |
| 5 | 10 | 5 |
| 1 | 5 | 1.8 |
| 3 | 5 | 1.8 |
| 5 | 3 | 1.8 |
| 1 | 10 | 5.4 |
| 10 | 1 | 4.2 |
| 1 | 3 | 5.3 |
| 3 | 1 | 4 |
| 3 | 10 | No Pacing |
| 10 | 3 | No Pacing |

As can be seen, successful ventricular pacing was achieved at all pairs of Angular Locations except the pair of Angular Locations 3 and 10, with both polarities. The best pacing, as indicated by the minimum voltage required to achieve pacing, was achieved at the following pairs of Angular Locations (Anode-Cathode, respectively): 10-5, 1-5, 3-5, and 5-3.

As can be seen, the best ventricular pacing, as indicated by the minimum voltage required to achieve ventricular pacing, was achieved using arcs that included Angular Location 5 and/or Angular Location 1, both positioned near the bundle of His, with Angular Location 5 providing lower pacing thresholds. Better pacing with any given pair of electrodes was achieved when the cathode was placed at Angular Location 5 or Angular Location 1 than when the anode was placed at this location.

No degradation of performance was observed after pacing from a certain point multiple times.

### Experiment #4

A fourth experiment conducted by the inventors was designed to assess whether cardiac ventricular pacing could be achieved using cathodes disposed at various electrode locations on and above the LVOT. This experiment was designed to assess feasibility of pacing by stimulation at various points in and around the heart, as described hereinabove with reference to FIG. 7B. Cathode leads and anode leads were assembled on semi-rigid rods and placed at the locations set forth in Table 11 below in a pig having a weight of 90 kg. Intracardiac and intra-aortic locations were reached via a transapical sheath. Pacing was attempted, each time at a different electrode location, and whether pacing was achieved was recorded. A Medtronic 5348 temporary pulse generator was used (Medtronic, Minneapolis, Minnesota).

Table 11 presents ventricular pacing results of Experiment #4:

TABLE 11

| | | Pacing |
|---|---|---|
| Anode Location | Cathode Location | achieved? |
| Within the trans-apical | Within the trans-apical sheath | No |
| sheath | | |
| In LVOT against atrio- | On the pericardium | Yes |
| ventricular wall | | |
| In LV, no contact with | LV, no contact with endocardium | No |
| endocardium | | |
| In the LV on the | In the LV space, no tissue contact | No |
| endocardium | | |
| Against aortic root | In the lv space, no tissue contact | No |
| wall | | |
| In the Aortic root | In the LV against the endocardium | No |
| without tissue contact | | |
| Touching the aortic | Between the NCC-RCC commissure | Yes |
| root wall | and aortic annulus plane 110 | |
| Touching the aortic | Between the NCC-LCC commissure | Yes |
| root wall | and aortic annulus plane 110 | |
| Touching the aortic | Between the RCC-LCC commissure | No |
| root wall | and aortic annulus plane 110 | |
| At annulus on anterior | Between the NCC-RCC commissure | Yes |
| side of valve | and annulus plane 110 | |
| Touching the aortic | Touching the aortic root wall | No |
| root wall | | |

* Position of the electrode was estimated based on the imaging capabilities at hand but could not be precisely verified.

As can be seen, successful ventricular pacing was achieved when the cathode was in contact with the interleaflet triangle between the NCC-RCC commissure and aortic annulus plane 310, and with the interleaflet triangle between the NCC-LCC commissure and aortic annulus plane 310, but not with the interleaflet triangle between the RCC-LCC commissure and aortic annulus plane 310.

Successful ventricular pacing was also achieved when the cathode was in contact with the interleaflet triangle between the NCC-RCC commissure and aortic annulus plane 310 and the anode was at the annulus level against the anterior side of the aortic valve.

### Experiment #5

A fifth experiment conducted by the inventors was designed, among other things, to assess the minimal pacing voltage required to achieve ventricular or atrial pacing using electrodes disposed at various electrode locations on an external surface of a frame of a prosthetic aortic valve similar to prosthetic aortic valve 20 described hereinabove with reference to FIGS. 1A-3I. Other than as described below, the fifth experiment was conducted in a similar manner to the horizontal pacing portion of Experiment #2 described hereinabove, and in a similar manner to Experiment #3 described hereinabove. The prosthetic aortic valve had a diameter of 27 mm and included eight electrodes disposed at various respective angular locations with respect to a central longitudinal axis of the frame. The prosthetic aortic valve was implanted transapically in the native aortic valve of four pigs having a weight of 100±5 kg.

The results of a first portion of Experiment #5 are summarized schematically in FIGS. 9B and 9C, in which the pacing locations of this experiment are schematically indicated by circled numbers 1, 3, 5, 7, 8, 9, 11, and 12, using the numbers for the angular locations set forth hereinabove in Table 1. FIG. 9B shows pacing locations that achieved ventricular pacing with zero activation time (stimulus-to-QRS interval), and FIG. 9C shows pacing locations that achieved ventricular pacing with delayed activation). The pairs that successfully achieved ventricular pacing are labeled in FIG. 9B or FIG. 9C, and the pairs that achieved atrial pacing are labeled in FIG. 9D.

FIGS. 9B and 9C also show one set of representative ECG and intracardiac EGM traces (ECG and intracardiac EGM traces were produced for the other pacing locations as well). The ECG traces were acquired conventionally, using external ECG skin electrodes on the chest. The intracardiac EGM trace shown in FIG. 9B was acquired using a pair of electrodes at Anode Angular Location 11 and Cathode Angular Location 7 during pacing between Anode Angular Location 5 and Cathode Angular Location 12. The intracardiac EGM trace shown in FIG. 9C was acquired using a pair of electrodes at Anode Angular Location 1 and Cathode Angular Location 5 during pacing between Anode Angular Location 12 and Cathode Angular Location 3.

Each pair of electrodes included an anode and a cathode, both of which were disposed sub-annularly, approximately 4-6 mm below the annulus, at two different angular locations. As in Experiments #1, #2, and #4, the electrodes were directly, non-wirelessly connected to the pulse generator. The system enabled, by wireless commands from the external transmitter, configuration of each electrode to be a cathode or an anode.

Each pair of electrodes is schematically labeled in FIGS. 9B-C by a connecting line, the ends of which indicate the angular locations of the anode and the cathode. As indicated in the key in FIGS. 9B-C, the endpoints of the lines indicate which electrode was an anode (by a circle) and which electrode was a cathode (by a square). (The lines connect the electrode pairs only schematically, and may or may not indicate the actual conduction paths between the electrode pairs, which were not measured in the experiment.)

The following Table 12 summarizes the results for each of the pairs of Angular Locations, using the numbers set forth in Table 1 hereinabove. Only the pairs that successfully achieved ventricular pacing are labeled in FIG. 9B or FIG. 9C, while the pairs that successfully achieved atrial pacing are labeled in FIG. 9D, described hereinbelow.

TABLE 12

| Anode | Cathode | Min. | Type of |
|---|---|---|---|
| Angular | Angular | Voltage For | Pacing |
| Location | Location | Pacing [V] | Induced* |
| 9 | 11 | 7** | II |
| 9 | 12 | 7** | I |
| 5 | 9 | 8** | II |
| 5 | 1 | 6 | II |
| 5 | 12 | 6 | I |
| 7 | 11 | 10 | A |
| 3 | 8 | 8.5 | I |
| 3 | 7 | 3.6 | II |
| 3 | 12 | 5.6 | II |
| 1 | 7 | 4 | A |
| 9 | 7 | 3.6 | I |
| 8 | 7 | 3.8 | II |
| 8 | 1 | 1.4 | II |
| 3 | 5 | 1.4 | I |
| 7 | 5 | 1.4 | I |

*I = zero activation time; II = delayed activation; A = atrial pacing.
**Unlike the other voltages set forth in Table 12, the voltages labeled with an asterisk do not represent the minimum voltage capable of causing pacing, but instead a single voltage that was tested and resulted in pacing.

Reference is now made to FIGS. 10A-B, which are schematic illustrations showing results of an additional experiment conducted by the inventors, using techniques similar to some of those described herein. Any of the techniques described in this experiment may optionally be incorporated into the techniques of the inventions described herein, as appropriate.

### Experiment #6

A sixth experiment conducted by the inventors was designed to assess the feasibility of accurate sensing of a ventricular activation signal in an intracardiac EGM sensed using two of the electrodes disposed at various electrode locations on an external surface of a frame of a prosthetic aortic valve similar to prosthetic aortic valve 20 described hereinabove with reference to FIGS. 1A-3I. Experiment #6 was performed together with Experiment #5, described hereinabove, using the same experimental procedure.

A portion of the results of Experiment #6 are summarized schematically in FIGS. 10A-B, in which the sensing locations of this experiment are schematically indicated by circled numbers, using the numbers for the angular locations set forth hereinabove in Table 1. Table 13 below lists all of the pairs shown in FIGS. 10A-B.

Each pair of electrodes included an anode and a cathode, both of which were disposed sub-annularly, approximately 4-6 mm below the annulus, at two different angular locations.

Each pair of electrodes is schematically labeled in FIGS. 10A-B by a connecting line, the ends of which indicate the angular locations of the anode and the cathode. As indicated in the key in FIGS. 10A-B, the endpoints of the lines indicate which electrode was an anode (by a circle) and which electrode was a cathode (by a square). (The lines connect the electrode pairs only schematically, and may or may not indicate the actual conduction paths between the electrode pairs, which were not measured in the experiment.)

Sensing was attempted of the heart's natural electrical activity, without pacing, each time through a different pair of electrodes.

For each pair of electrodes, an intracardiac EGM was measured and the voltage of the highest amplitude component (as an average of all the spikes) of the signal was identified as representing the ventricular activation signal. The second-highest amplitude component (generally as an average of all the spikes) of the signal was also identified, and the ratio between the voltage of the highest amplitude component and the voltage of the second-highest amplitude component was calculated. If the highest amplitude component had a positive voltage, the next-highest amplitude component with a positive voltage was identified; if the highest amplitude component had a negative voltage, the next-highest amplitude component with a negative voltage was identified.

The following Table 13 summarizes the results for each of the pairs of Angular Locations, using the numbers set forth in Table 1 hereinabove, including a V-Signal Separation Ratio (VSR), which is the ratio between the ventricular component and the second-highest component of the signal:

TABLE 13

| Anode | Cathode | Ventricular | | |
|---|---|---|---|---|
| angular | angular | activation | Second-highest | |
| location | location | signal [mV] | component [mV] | VSR |
| 11 | 5 | 1.2 | 0.31 | 3.9 |
| 11 | 9 | −1.13 | −0.21 | 5.4 |
| 1 | 12 | −1.6 | −0.19 | 8.7 |
| 11 | 7 | 0.98 | 0.22 | 3.3 |
| 3 | 7 | 1.66 | 0.25 | 6.6 |
| 8 | 12 | 1.6 | 0.41 | 4.0 |
| 8 | 9 | 1.66* | 0.54 | 3.1 |
| 8 | 3 | 0.43 | 0.28 | 1.5 |
| 12 | 3 | 1.67* | 0.29 | 5.8 |
| 1 | 3 | −1.67* | −0.28 | 6.0 |
| 5 | 12 | −1.66* | −0.27 | 6.1 |
| 5 | 7 | 1.19 | 0.48 | 2.7 |
| 1 | 11 | −0.54 | −0.43 | 1.3 |
| 5 | 8 | −1.66* | −0.3 | 5.5 |
| 7 | 9 | 1.66* | 0.44 | 3.8 |
| 9 | 7 | 1.66* | 0.47 | 3.5 |
| 5 | 3 | −1.66* | −0.14 | 11.9 |

*Technological constraints in this experiment limited the measured EGM voltage to between −1.66 or −1.67 and +1.66 mV. Actual values may be higher.

As can be seen in Table 13, in all of the tested pairs the ventricular signal was distinct and significantly higher than the second-highest component of the signal, except for one acquisition through electrodes at Anode Angular Location 1 and Cathode Angular Location 11, and for one acquisition through electrodes at Anode Angular Location 8 and Cathode Angular Location 3, in which the ventricular signal was only modestly higher than the second-highest component of the signal.

### Experiment #7

A seventh experiment conducted by the inventors was designed, among other things, to assess the minimal pacing voltage required to achieve atrial pacing using electrodes disposed at various electrode locations on an external surface of a frame of a prosthetic aortic valve similar to prosthetic aortic valve 20 described hereinabove with reference to FIGS. 1A-3I. Other than as described below, the seventh experiment was conducted in a similar manner to the horizontal pacing portion of Experiment #2 described hereinabove, and in a similar manner to Experiment #3 described hereinabove, except that the electrodes in Experiment #7 comprised gold-plated copper pads. The prosthetic aortic valve had a diameter of 27 mm and included eight electrodes disposed at various respective angular locations with respect to a central longitudinal axis of the frame. The prosthetic aortic valve was implanted transapically in the native aortic valve of one pig having a weight of 100 kg.

The results of Experiment #7 that relate to atrial pacing are summarized schematically in FIG. 9D (in which a portion of the results of Experiment #5 are also shown, as described above), in which the pacing locations of this experiment are schematically indicated by circled numbers 10 and 11, using the numbers for the angular locations set forth hereinabove in Table 1. FIG. 9D shows a pacing location that achieved atrial pacing. This atrial pacing was achieved using a minimum voltage of 14 V. FIG. 9D also shows an ECG trace for the pacing applied in Experiment #7. The ECG trace was acquired conventionally, using external ECG skin electrodes on the chest.

The pair of pacing electrodes included an anode and a cathode, both of which were disposed sub-annularly, approximately 4-6 mm below the annulus, at Anode Angular Location 10 and Cathode Angular Location 11, respectively. As in Experiments #1, #2, #4, and #5, the electrodes were directly, non-wirelessly connected to the pulse generator.

The pair of pacing electrodes is schematically labeled in FIG. 9D by a connecting line, the ends of which indicate the angular locations of the anode and the cathode. As indicated in the key in FIG. 9D, the endpoints of the lines indicate which electrode was an anode (by a circle) and which electrode was a cathode (by a square). (The lines connect the electrode pairs only schematically, and may or may not indicate the actual conduction paths between the electrode pairs, which were not measured in the experiment.)

A portion of Experiment #7 was designed to assess the feasibility of accurate sensing of a ventricular activation signal and an atrial activation signal in an intracardiac EGM sensed using two of the electrodes disposed at various electrode locations on the external surface of the frame of the prosthetic aortic valve, without applying pacing. Successful sensing of ventricular activation signals and atrial activation signals was achieved using multiple pairs of electrodes. For example, as schematically labeled in FIG. 9D, both a ventricular intrinsic activation signal (labeled ‘V’ in the intracardiac EGM) and an atrial intrinsic activation signal (labeled ‘A’ in the intracardiac EGM) were successfully identified in an intracardiac EGM sensed using electrodes disposed at Anode Angular Location 10 and Cathode Angular Location 12 (without application of pacing).

### Experiment #8

An eighth experiment conducted by the inventors was designed, among other things, to assess the minimal pacing voltage required to achieve ventricular pacing using electrodes disposed at various electrode locations on an external surface of a frame of a prosthetic aortic valve similar to prosthetic aortic valve 20 described hereinabove with reference to FIGS. 1A-3I. Other than as described below, the eighth experiment was conducted in a similar manner to the horizontal pacing portion of Experiment #2 described hereinabove, and in a similar manner to Experiment #3 described hereinabove. The prosthetic aortic valve had a diameter of 27 mm and included eight electrodes disposed at various respective angular locations with respect to a central longitudinal axis of the frame. The prosthetic aortic valve was implanted transapically in the native aortic valve of two pigs having a weight of 95-100 kg.

Each pair of electrodes included an anode and a cathode, both of which were disposed sub-annularly, approximately 4-6 mm below the annulus, at two different angular locations. As in Experiments #1, #2, and #4, the electrodes were directly, non-wirelessly connected to the pulse generator. The system enabled, by wireless commands from the external transmitter, configuration of each electrode to be a cathode or an anode.

The following Table 14 summarizes the results for each of the pairs of Angular Locations, using the numbers set forth in Table 1 hereinabove:

TABLE 14

| | | Min. | | | Min. |
|---|---|---|---|---|---|
| Cathode | Anode | Voltage | Cathode | Anode | Voltage |
| Angular | Angular | For | Angular | Angular | For |
| Location | Location | Pacing [V] | Location | Location | Pacing [V] |
| 10 | 13 | 14 | 4 | 10 | 1.8 |
| 10 | 12 | 12 | 4 | 3 | 1.8 |
| 10 | 4 | 16 | 4 | 13 | 2 |
| 10 | 5 | 7.5 | 4 | 12 | 2 |
| 10 | 6 | 18 | 4 | 5 | 1.8 |
| 10 | 11 | N* | 4 | 6 | 2 |
| 3 | 13 | 16 | 4 | 11 | 4 |
| 3 | 12 | 10 | 5 | 10 | 2.4 |
| 3 | 4 | 2.4 | 5 | 3 | 2.6 |
| 3 | 5 | 8 | 5 | 13 | 1.9 |
| 3 | 6 | 5.4 | 5 | 12 | 2.6 |
| 3 | 11 | N | 5 | 4 | 1.8 |
| 13 | 10 | 0.8 | 5 | 6 | 2.8 |
| 13 | 3 | 0.8 | 5 | 11 | 3 |
| 13 | 12 | 1 | 6 | 10 | 3.2 |
| 13 | 4 | 1 | 6 | 3 | 3.4 |
| 13 | 5 | 1 | 6 | 13 | 3.4 |
| 13 | 6 | 1 | 6 | 12 | 3.6 |
| 13 | 11 | 1 | 6 | 4 | 2.4 |
| 12 | 10 | 1.6 | 6 | 5 | 3.2 |
| 12 | 3 | 1.8 | 6 | 11 | 3 |
| 12 | 13 | 2 | 11 | 10 | 16 |
| 12 | 4 | 1.8 | 11 | 3 | 16 |
| 12 | 5 | 1.6 | 11 | 13 | 2.6 |
| 12 | 6 | 2.6 | 11 | 12 | 5 |
| 12 | 11 | 1.8 | 11 | 4 | 2.4 |
| | | | 11 | 5 | 9 |
| | | | 11 | 6 | 4.4 |

*N = no pacing

As can be seen, in general (with a few exceptions), Cathode Angular Locations 4, 5, 6, 11, 12, and 13 had lower minimum pacing voltages that Cathode Angular Locations 3 and 10, including for some of the same pairs of Angular Locations at which the polarities were also reversed. As can also be seen, stimulations using a cathode located in the segment stretching between Cathode Angular Locations 5 and 13 yielded the lowest threshold. The inventors observed that the pacing threshold correlates better with the cathode location than with the anode location; in other words, the cathode location is a stronger determinant of the pacing threshold than is the anode location.

### Experiment #9

A seventh experiment conducted by the inventors was designed to assess both:
the minimal pacing voltage required to achieve pacing using two of the electrodes disposed at various electrode locations on an external surface of a frame of a prosthetic aortic valve similar to prosthetic aortic valve 20 described hereinabove with reference to FIGS. 1A-3I, and
the feasibility of accurate sensing of a ventricular activation signal in an intracardiac EGM sensed, during application of pacing, using two of the electrodes disposed at various electrode locations on an external surface of a frame of a prosthetic aortic valve similar to prosthetic aortic valve 20 described hereinabove with reference to FIGS. 1A-3I.

Experiment #9 was performed together with Experiment #5, described hereinabove, using the same experimental procedure.

The results of Experiment #9 are summarized schematically in FIGS. 11A-E, in which the pacing and sensing locations of this experiment are schematically indicated by circled numbers, using the numbers for the angular locations set forth hereinabove in Table 1. Only a subset of the electrode pairs tested are labeled in FIGS. 11A-E, while all the tested pairs are listed in Table 15 below.

Each pair of electrodes included an anode and a cathode, both of which were disposed sub-annularly, approximately 4-6 mm below the annulus, at two different angular locations.

Each pair of electrodes is schematically labeled in FIGS. 11A-E by a connecting line. (The lines connect the electrode pairs only schematically, and may or may not indicate the actual conduction paths between the electrode pairs, which were not measured in the experiment.) FIGS. 11A-E also show ECGs, measured using external skin electrodes, and intracardiac EGMs, measured using the respective pairs of electrodes.

Pacing was performed and assessed in a manner similar to the horizontal pacing portion of Experiment #2, and Experiments #3 and #5, all described hereinabove. Sensing was assessed in a similar manner to Experiment #6, described hereinabove. Intracardiac EGM samples taken in this trial were 15 seconds long, at a 90 Hz acquisition rate (the intracardiac EGM was sampled discretely for a brief instance once every 11 milliseconds). The pacing rate was typically 100 bpm.

The following Table 15 summarizes the results for each of the pairs of Angular Locations, using the numbers set forth in Table 1 hereinabove, including a V-Signal Separation Ratio (VSR), which is the ratio between the ventricular component and the second-highest component of the signal:

TABLE 15

| PACING | | | SENSING | | | |
|---|---|---|---|---|---|---|
| Anode | Cathode | Pacing | Anode | Cathode | Ventricular | |
| angular | angular | voltage | angular | angular | activation | |
| location | location | [V] | location | location | signal [mV] | VSR |
| 9 | 11 | 7 | 1 | 5 | 0.39 | 1.70 |
| 9 | 12 | 7 | 1 | 5 | −1.56 | 4.59 |
| 9 | 12 | 8 | 1 | 5 | −1.43 | 6.22 |
| 5 | 9 | 8 | 7 | 11 | 0.71 | 1.26 |
| 5 | 1 | 8 | 7 | 11 | 0.47 | 0.89 |
| 5 | 1 | 7 | 7 | 11 | 0.58 | 1.29 |
| 5 | 12 | 6 | 7 | 11 | 0.14 | 0.93 |
| 5 | 12 | 4 | 7 | 11 | 0.88 | 2.84 |
| 5 | 12 | 2 | 7 | 11 | 1.67 | 5.35 |
| 7 | 11 | 10 | 5 | 1 | 0.88 | 1.83 |
| 3 | 8 | 8.5 | 5 | 1 | 1.67 | 3.33 |
| 3 | 7 | 3.6 | 5 | 1 | 0.67 | 3.19 |
| 3 | 12 | 5.6 | 5 | 1 | 1.67 | 4.39 |
| 1 | 7 | 4 | 12 | 9 | −1.54 | 1.81 |
| 9 | 7 | 3.6 | 12 | 8 | −1.43 | 3.25 |
| 8 | 7 | 3.8 | 9 | 12 | | §§ |
| 3 | 5 | 1.4 | 9 | 12 | | §§ |
| 7 | 5 | 1.4 | 5 | 7 | 0.87 | 2.71 |

§ Based on the VSR without T wave exclusion. H: VSR > 2.2; M: 2.2 > VSR > 1.8; L: 1.8 > VSR
§§ Chaotic (although good capture was visible in the ECG, the intracardiac EGM signal was noisy to the extent that V signals could not be distinguished from the remainder of the signal)

In addition, using the pair of Anode angular location 7 and Cathode angular location 11, atrial pacing was triggered and atrial signals were observed in the EGM.

In an embodiment, techniques and apparatus described in one or more of the following patents and/or applications, which are assigned to the assignee of the present application and are incorporated herein by reference, are combined with techniques and apparatus described herein:
U.S. Pat. No. 10,543,083 to Gross
European Patent Application Publication EP 3508113 A1 to Gross
U.S. Pat. No. 10,835,750 to Gross
U.S. Pat. No. 11,013,597 to Gross
PCT Publication WO 2021/140507 to Gross
PCT Publication WO 2021/224904 to Gross
U.S. Pat. No. 11,065,451 to Gross
U.S. Pat. No. 11,291,844 to Gross
PCT Publication WO 2022/149130 to Gross
U.S. Pat. No. 11,931,255 to Gross et al.
U.S. Pat. No. 11,975,203 to Gross et al.
US Patent Application Publication 2025/0058124 to Gross et al.
PCT Publication WO 2025/041129 to Gross et al.
U.S. Provisional Application 63/717,923, filed Nov. 8, 2024
U.S. Provisional Application 63/760,353, filed Feb. 19, 2025
U.S. application Ser. No. 19/068,620, filed Mar. 3, 2025
U.S. Provisional Application 63/809,535, filed May 21, 2025
U.S. Provisional Application 63/849,229, filed Jul. 23, 2025
U.S. Provisional Application 63/849,212, filed Jul. 23, 2025
U.S. application Ser. No. 19/381,343, filed Nov. 6, 2025
U.S. application Ser. No. 19/409,867, filed Dec. 5, 2025

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A prosthetic cardiac valve system comprising a prosthetic cardiac valve, which is configured to be delivered to a native cardiac valve of a heart of a patient in a constrained delivery configuration, and which comprises:
a frame, which comprises interconnected stent cells, which include distal stent cells that are located in a distal half of the frame and are shaped so as to define respective distal peaks;
a plurality of prosthetic leaflets coupled to the frame so as to allow blood flow in a downstream direction and inhibit blood flow in an upstream direction when the prosthetic cardiac valve is in an expanded deployment configuration;
electrodes, which include a plurality of distal electrodes mechanically coupled to the frame at or near respective ones of the distal peaks; and
circuitry, which is electrically coupled to the electrodes, and which is configured to apply pacing to the heart using one or more of the distal electrodes as one or more anodes and one or more of the other distal electrodes as one or more cathodes.

2. The prosthetic cardiac valve system according to claim 1, wherein the circuitry is configured to apply ventricular pacing to the heart using the one or more of the distal electrodes as the one or more anodes and the one or more of the other distal electrodes as the one or more cathodes.

3. The prosthetic cardiac valve system according to claim 1, wherein the circuitry is configured to apply atrial pacing to the heart using the one or more of the distal electrodes as the one or more anodes and the one or more of the other distal electrodes as the one or more cathodes.

4. The prosthetic cardiac valve system according to claim 1, wherein the distal electrodes are disposed at a common axial position along a central longitudinal axis of the frame when the prosthetic cardiac valve is in the expanded deployment configuration.

5. The prosthetic cardiac valve system according to claim 1, wherein the circuitry is configured to apply the pacing to the heart using exactly one of the distal electrodes as an anode and exactly one of the other distal electrodes as a cathode.

6. The prosthetic cardiac valve system according to claim 1, wherein the circuitry is configured to apply the pacing to the heart using the one or more of the distal electrodes as the one or more anodes and two or more of the other distal electrodes as two or more cathodes.

7. The prosthetic cardiac valve system according to claim 6, wherein the circuitry is configured to apply the pacing to the heart using exactly one of the distal electrodes as an anode and the two or more of the other distal electrodes as the two or more cathodes.

8. The prosthetic cardiac valve system according to claim 1, wherein the distal electrodes are mechanically coupled to the frame at or within 8 mm of the respective ones of the distal peaks.

9. The prosthetic cardiac valve system according to claim 1, wherein the distal electrodes are mechanically coupled to the frame at the respective ones of the distal peaks.

10. The prosthetic cardiac valve system according to claim 1, wherein the distal electrodes are mechanically coupled to the frame at or near respective distal peaks of distal-most ones of the distal stent cells.

11. The prosthetic cardiac valve system according to claim 1, wherein the prosthetic cardiac valve is a prosthetic aortic valve.

12. The prosthetic cardiac valve system according to claim 11, wherein the distal peaks are respective upstream distal peaks, and wherein the distal electrodes are upstream distal electrodes that are mechanically coupled to the frame at or near respective ones of the upstream distal peaks.

13. The prosthetic cardiac valve system according to claim 1, wherein the prosthetic cardiac valve is a prosthetic atrioventricular valve.

14. The prosthetic cardiac valve system according to claim 13, wherein the distal peaks are respective downstream distal peaks, and wherein the distal electrodes are downstream distal electrodes that are mechanically coupled to the frame at or near respective ones of the downstream distal peaks.

15. A prosthetic cardiac valve system comprising a prosthetic cardiac valve, which is configured to be delivered to a native cardiac valve of a heart of a patient in a constrained delivery configuration, and which comprises:
a frame, which comprises interconnected stent cells, which include distal stent cells that are located in a distal half of the frame and are shaped so as to define respective distal peaks;
a plurality of prosthetic leaflets coupled to the frame so as to allow blood flow in a downstream direction and inhibit blood flow in an upstream direction when the prosthetic cardiac valve is in an expanded deployment configuration;
electrodes, which include a plurality of distal electrodes mechanically coupled to the frame at or near respective ones of the distal peaks; and
circuitry, which is electrically coupled to the electrodes, and which is configured to sense cardiac electrical activity of the patient using one or more of the distal electrodes as one or more anodes and one or more of the other distal electrodes as one or more cathodes.

16. The prosthetic cardiac valve system according to claim 15, wherein the circuitry is configured to sense an intracardiac electrogram (EGM) of the heart using the one or more of the distal electrodes as the one or more anodes and the one or more of the other distal electrodes as the one or more cathodes.

17. The prosthetic cardiac valve system according to claim 15, wherein the distal electrodes are disposed at a common axial position along a central longitudinal axis of the frame when the prosthetic cardiac valve is in the expanded deployment configuration.

18. The prosthetic cardiac valve system according to claim 15, wherein the circuitry is configured to sense the cardiac electrical activity using exactly one of the distal electrodes as an anode and exactly one of the other distal electrodes as a cathode.

19. The prosthetic cardiac valve system according to claim 15, wherein the distal electrodes are mechanically coupled to the frame at or within 8 mm of the respective ones of the distal peaks.

20. The prosthetic cardiac valve system according to claim 15, wherein the distal electrodes are mechanically coupled to the frame at the respective ones of the distal peaks.

21. The prosthetic cardiac valve system according to claim 15, wherein the distal electrodes are mechanically coupled to the frame at or near respective distal peaks of distal-most ones of the distal stent cells.

22. The prosthetic cardiac valve system according to claim 15, wherein the prosthetic cardiac valve is a prosthetic aortic valve.

23. The prosthetic cardiac valve system according to claim 22, wherein the distal peaks are respective upstream distal peaks, and wherein the distal electrodes are upstream distal electrodes that are mechanically coupled to the frame at or near respective ones of the upstream distal peaks.

24. The prosthetic cardiac valve system according to claim 15, wherein the prosthetic cardiac valve is a prosthetic atrioventricular valve.

25. The prosthetic cardiac valve system according to claim 24, wherein the distal peaks are respective downstream distal peaks, and wherein the distal electrodes are downstream distal electrodes that are mechanically coupled to the frame at or near respective ones of the downstream distal peaks.
